(19) United States
(12) Patent Application Publication
(10) Pub. No.: US 2022/0062317 A1
(43) Pub. Date: Mar. 3, 2022

(54) NA+/K+ ATPASE INHIBITORS FOR USE IN THE PREVENTION OR TREATMENT OF METASTASIS

(71) Applicant: UNIVERSITÄT BASEL, Basel (CH)

(72) Inventors: Nicola ACETO, Basel (CH); Sofia GKOUNTELA, Basel (CH)

(73) Assignee: UNIVERSITÄT BASEL, Basel (CH)

(21) Appl. No.: 17/415,735

(22) PCT Filed: Dec. 20, 2019
(86) PCT No.: PCT/EP2019/086633

§ 371 Date: Jun. 18, 2021

(30) Foreign Application Priority Data
Dec. 20, 2018 (EP) 18214978.1

(51) Int. Cl. A61K 31/7048; A61K 31/585; A61K 31/7105; A61P 35/00

(52) U.S. Cl. CPC A61K 31/7048; A61P 35/00; A61K 31/7105; A61K 31/585

(57) ABSTRACT

The invention relates to an Na⁺/K⁺ ATPase inhibitor for use in the prevention or treatment of metastasis in a cancer patient defined by the presence of CTC clusters in the bloodstream. In certain embodiments the Na⁺/K⁺ ATPase is a cardiac glycoside and is selected from: digitoxin, ouabain, convallatoxin, proscillaridin, lanatoside C, gitoformate, peruvoside, strophanthidin, metildigoxin, deslanoside, bufalin, digoxin and digoxigenin. The invention further relates to the use of nucleic acid agents inhibiting the expression of genes related to CTC cluster formation and maintenance.

The present invention relates to Na⁺/K⁺ ATPase inhibitors for use in the prevention or treatment of metastasis.

This application claims the benefit of the priority of European patent application EP18214978.1 filed 20 Dec. 2018, which is incorporated herein in its entirety.

### BACKGROUND

Metastatic spread of cancer, typically to bone, lung, liver and brain, accounts for the vast majority of cancer-related deaths. Epithelial cancer metastasis is thought to involve a series of sequential steps: epithelial-to-mesenchymal transition (EMT) of individual cells within the primary tumor leading to their intravasation into the bloodstream, survival of such circulating tumor cells (CTCs) within the bloodstream, and finally their extravasation at distant sites, where mesenchymal-to-epithelial transition (MET) culminates in their proliferation as epithelial metastatic deposits.

Circulating tumor cells are cells that depart from a cancerous tumor and enter the bloodstream, on their way to seeding metastasis (Alix-Panabieres et. al., Clin Chem 59, 110-118, 2013). The analysis of CTCs holds the great promise to dissecting those fundamental features of the metastatic process, enabling the identification of targetable cancer vulnerabilities. Once in the bloodstream, to survive, CTCs need to overcome the loss of adhesion signals from the primary tumor as well as high shear forces that are proper of the circulatory system. In breast cancer, the ability of CTCs to form clusters has been linked to increased metastatic propensity when compared to single CTCs (Aceto et al.; Cell 158, 1110-1122, 2014).

CTCs are found in the blood of cancer patients as single CTCs and CTC clusters (Fidler European Journal of Cancer 9, 223-227 1973; Liotta et al., Cancer Research 36, 889-894 1976), with the latter featuring a higher ability to seed metastasis (Aceto et al. Cell 158, 1110-1122, 2014). Yet, what drives their enhanced metastatic potential and what are the vulnerabilities of clustered CTCs is unknown.

Based on the above-mentioned state of the art, the objective of the present invention is to provide means and methods to prevent and treat metastasis in cancer patients. This objective is attained by the claims of the present specification.

### DESCRIPTION

### Summary of the Invention

The inventors profiled the DNA methylation landscape of single CTCs and CTC-clusters at genome-wide scale, matched within individual cancer patients and human CTC-derived xenografts. They surprisingly found that stemness-related transcription factors orchestrate an OCT4-centric network that is exclusively active in CTC-clusters, and that simultaneously CTC clusters display activation of a SIN3A-dependent cell cycle progression program. This finding demonstrates that the ability of CTCs to form clusters directly impacts on their DNA methylation pattern and results in enhanced stemness and cell cycle progression signals that favor metastasis seeding.

The inventors identified drugs that specifically disrupt CTC-clusters without altering their cellular viability. Upon cluster disruption into single cells, DNA methylation is re-gained at critical sites to shut down the clustering-associated stemness and cell cycle programs, leading to a significant reduction in metastasis-seeding ability.

A first aspect of the invention relates to an Na⁺/K⁺ ATPase inhibitor for use in the prevention or treatment of metastasis in a cancer patient.

A second aspect of the invention relates to nucleic acid mediated therapeutic downregulation or inhibition expression of a target nucleic acid sequence encoding a protein selected from:
CLDN3,
CLDN4 and
Na+/K+ ATPase or any of its constituent subunit isoforms.

A third aspect of the invention relates to the use of an Na⁺/K⁺ ATPase inhibitor or a nucleic acid molecule according to the invention in the prevention and treatment of venous thromboembolism in cancer patients.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The terms capable of forming a hybrid or hybridizing sequence in the context of the present specification relate to sequences that under the conditions existing within the cytosol of a mammalian cell, are able to bind selectively to their target sequence. Such hybridizing sequences may be contiguously reverse-complimentary to the target sequence, or may comprise gaps, mismatches or additional non-matching nucleotides. The minimal length for a sequence to be capable of forming a hybrid depends on its composition, with C or G nucleotides contributing more to the energy of binding than A or T/U nucleotides, and the backbone chemistry.

The term Nucleotides in the context of the present specification relates to nucleic acid or nucleic acid analogue building blocks, oligomers of which are capable of forming selective hybrids with RNA or DNA oligomers on the basis of base pairing. The term nucleotides in this context includes the classic ribonucleotide building blocks adenosine, guanosine, uridine (and ribosylthymine), cytidine, the classic deoxyribonucleotides deoxyadenosine, deoxyguanosine, thymidine, deoxyuridine and deoxycytidine. It further includes analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). Wherever reference is made herein to a hybridizing sequence, such hybridizing sequence may be composed of any of the above nucleotides, or mixtures thereof.

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The terms gene expression or alternatively gene product refer to the processes—and products thereof—of nucleic acids (RNA) or amino acids (e.g., peptide or polypeptide) being generated when a gene is transcribed and translated.

As used herein, expression refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell.

The term antisense oligonucleotide in the context of the present specification relates to an oligonucleotide having a sequence substantially complimentary to, and capable of hybridizing to, an RNA. Antisense action on such RNA will lead to modulation, particular inhibition or suppression of the RNA's biological effect. If the RNA is an mRNA, expression of the resulting gene product is inhibited or suppressed. Antisense oligonucleotides can consist of DNA, RNA, nucleotide analogues and/or mixtures thereof. The skilled person is aware of a variety of commercial and non-commercial sources for computation of a theoretically optimal antisense sequence to a given target. Optimization can be performed both in terms of nucleobase sequence and in terms of backbone (ribo, deoxyribo, analogue) composition. Many sources exist for delivery of the actual physical oligonucleotide, which generally is synthesized by solid state synthesis.

The term siRNA (small/short interfering RNA) in the context of the present specification relates to an RNA molecule capable of interfering with the expression (in other words: inhibiting or preventing the expression) of a gene comprising a nucleic acid sequence complementary or hybridizing to the sequence of the siRNA in a process termed RNA interference. The term siRNA is meant to encompass both single stranded siRNA and double stranded siRNA. siRNA is usually characterized by a length of 17-24 nucleotides. Double stranded siRNA can be derived from longer double stranded RNA molecules (dsRNA). According to prevailing theory, the longer dsRNA is cleaved by an endo-ribonuclease (called Dicer) to form double stranded siRNA. In a nucleoprotein complex (called RISC), the double stranded siRNA is unwound to form single stranded siRNA. RNA interference often works via binding of an siRNA molecule to the mRNA molecule having a complementary sequence, resulting in degradation of the mRNA. RNA interference is also possible by binding of an siRNA molecule to an intronic sequence of a pre-mRNA (an immature, non-spliced mRNA) within the nucleus of a cell, resulting in degradation of the pre-mRNA.

The term shRNA (small hairpin RNA) in the context of the present specification relates to an artificial RNA molecule with a tight hairpin turn that can be used to silence target gene expression via RNA interference (RNAi).

The term sgRNA (single guide RNA) in the context of the present specification relates to an RNA molecule capable of sequence-specific repression of gene expression via the CRISPR (clustered regularly interspaced short palindromic repeats) mechanism.

The term miRNA (microRNA) in the context of the present specification relates to a small non-coding RNA molecule (containing about 22 nucleotides) that functions in RNA silencing and post-transcriptional regulation of gene expression.

The term inhibitor in the context of the present specification relates to a compound that is able to significantly reduce or completely abolish a physiologic function, activity or synthesis of a target molecule. On an abstract level, inhibition encompasses the interference with the biosynthesis of the target, the prevention of enzyme-substrate binding (the target being the substrate or the enzyme), the prevention of ligand-receptor interaction, etc.

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

In the context of the present specification, the term prevention or treatment of metastasis relates to the process of inhibiting the formation of new metastases that have not existed prior to treatment. This includes but is not limited to reducing the survival rate of cancer cells in the circulation, inhibiting of the extravasation of cancer cells from the blood stream and inhibiting of the seeding process at the site of extravasation.

### DETAILED DESCRIPTION OF THE INVENTION

A first aspect of the invention relates to an Na⁺/K⁺ ATPase inhibitor for use in the prevention or treatment of metastasis in a cancer patient. Any cancer patient, particularly in stages of the disease that lead an elevated risk of metastasis, may be considered at risk of developing metastatic disease mediated by or associated with the presence of CTC.

In particular embodiments, the Na⁺/K⁺ ATPase inhibitor is provided for treatment of cancer characterized by the presence of CTC clusters in the bloodstream. In such embodiments, the presence of CTC is a criterion for treatment according to the invention.

Na⁺/K⁺ ATPase is a transmembrane protein complex found in all higher eukaryotes acting as a key energy-consuming pump maintaining ionic and osmotic balance in cells. It is an enzyme (EC 3.6.3.9) that pumps sodium out of cells and potassium into cells. Both ions are actively pumped against their electrochemical gradient, expending energy in the form of ATP.

Na⁺/K⁺ ATPase is constituted of subunits, which may be targeted by antisense or other nucleic acid mediated intervention (e.g. CRISPR). Subunits are the alpha isoforms: ATP1A1 (alpha 1), ATP1A2 (alpha 2), ATP1A3 (alpha 3), and ATP1A4 (alpha 4) and the beta isoforms: ATP1B1 (beta 1), ATP1B2 (beta 2), ATP1B3 (beta 3) and ATP1B4 (beta 4). Intervention may target any subunit specifically, a combination of subunits based on shared sequence content, or all isoforms of the alpha and/or beta subunit based on identical mRNA sequence tracts.

In the particular context of the invention, an inhibitor of Na⁺/K⁺ ATPase significantly reduces or abolishes the target's enzymatic function, namely the pumping of sodium and potassium ions.

Exemplary inhibitors of Na⁺/K⁺ ATPase are known in different groups of chemical compounds. One group comprises well studied cardiac glycosides, including naturally occurring and synthetic inhibitors. Other examples of Na⁺/K⁺ ATPase inhibitors are steroidal Na⁺/K⁺ ATPase inhibitors such as androstenes and azaheterocyclyl derivatives of androstenes, in particular istaroxime (CAS 203737-93-3).

In certain embodiments, the inhibitor according to the invention reduces or prevents the formation of new metastasis. In certain embodiments, the inhibitor according to the invention is useful in the treatment of already existing metastasis. In certain embodiments, the inhibitor according to the invention is active in both prevention and treatment of metastasis.

Without wishing to be bound by theory, the inventors hypothesize that the Na⁺/K⁺ ATPase inhibitor for use in the prevention or treatment of metastasis according to the invention disrupts CTC clusters, resulting in single CTCs with a significantly decreased potential of metastasis formation as compared to CTC clusters. It is expected that cancer patients with CTC clusters in their bloodstream and/or an increased risk of CTC-clusters will benefit most from the inhibitors of the present invention. Since metastasis is associated with presence of CTC clusters and not in all situations, detection of CTC clusters will be possible, the treatments disclosed herein will be of benefit to any cancer patient suspected of being at risk of developing distant metastases from a primary tumour.

In certain embodiments, the inhibitors (or nucleic acid agents as described further below) are provided for use in breast cancer or prostate cancer.

Typically, patients with breast cancer and prostrate cancer have the highest incidence of CTC clusters. However, in all cancer types CTC clusters have been detected, therefore the Na⁺/K⁺ ATPase inhibitor of the current invention is expected to be beneficial to cancer patients in general.

The terms presence of CTC clusters in the bloodstream relates to cancer patients that have CTC clusters anywhere in their bloodstream. In particular large CTC-clusters might be difficult to detect in peripheral blood samples due to the fact that CTC-clusters are rapidly lodged in the capillary bed of blood vessels. Therefore, the absence of detectable CTC-clusters in peripheral blood samples is not necessarily an indicator for the absence of CTC-clusters everywhere in the bloodstream. Therefore, the skilled person is aware that the location of blood sampling for the detection of CTC-clusters might have to be chosen in dependence of the location of the primary tumor or metastasis that is shedding CTC-clusters.

Methods known to detect and/or isolate CTC clusters in blood samples include physical property-based methods that utilize differences in cell density, size, dielectric properties or mechanical plasticity. For example, a method based on size selection relies on the larger size of CTCs (and CTC clusters) in relation to other blood cells. A non-limiting example of a size based detection/isolation method is the use of the Parsortix device (Xu et al. PLoS One 10, e0138032, 2015). Another one was published by Shim et al. (Biomicrofluidics 2013, 7(1):11807 doi: 10.1063/1.4774304). In certain embodiments, the device for detection of CTC is a microfluidic device as disclosed in WO 2015/077603/US2016279637 (A1), or in WO2018005647 (A1)/US2019160464 (A1). In certain embodiments, the device is a microfluidic device as disclosed in US2014271909 (A1). Any of the patent documents cited herein are fully incorporated by reference.

Other known methods for the detection/isolation of CTC-clusters in cancer patients are antibody-based methods. The antibodies used are mainly specific to epithelial cell surface markers that are absent from blood or stroma cells. See also Balasubramanian et al. (PLoS 1 Apr. 12, 2017; https://doi.org/10.1371/journal.pone.0175414).

In the context of the present specification, the term circulating tumor cell (CTC) relates to cells that depart from a cancerous tumor and enter the bloodstream, on their way to seeding metastasis. CTCs can originate from a primary tumor as well as from an established metastasis. Therefore, the inhibitor of the present invention is useful in the treatment of cancer patients regardless of whether they already have an established metastasis or not.

In the context of the present specification, the term CTC cluster relates to aggregates of circulating tumor cells typically comprising 2 to 50 CTCs (Aceto et al., Cell 2014 ibid.).

The term “cancer”, as used herein, may be carcinoma including lung cancer, bladder cancer, breast cancer, colon cancer, renal cancer, rectal cancer, liver cancer, brain cancer, esophageal cancer, uterine cancer, gallbladder cancer, ovarian cancer, pancreatic cancer, stomach cancer, cervical cancer, thyroid cancer, prostate cancer, skin cancer, and hematopoietic tumors; tumors of mesenchymal origin, including fibrosarcoma and rhabdomyosarcoma; tumors of the central and peripheral nervous system, including astrocytoma, neuroblastoma, glioma and schwannomas; and other tumors, including melanoma, seminoma, teratocarcinoma, osteosarcoma, xeroderma pigmentosum, keratoctanthoma, thyroid follicular cancer and Kaposi's sarcoma, and particularly, prostate cancer, lung cancer, breast cancer, liver cancer, stomach cancer, renal cancer or uterine cancer.

In certain embodiments, the Na⁺/K⁺ ATPase inhibitor for use in the prevention or treatment of metastasis is for cancer patients with breast cancer or prostate cancer.

In certain embodiments, the cancer is a solid cancer. A solid cancer is characterized by a tumor that does not contain cysts or liquid areas.

In certain embodiments, the Na⁺/K⁺ ATPase inhibitor for use in the prevention or treatment of metastasis is a cardiac glycoside.

In the context of the present specification, the term cardiac glycoside relates to an organic compound that comprises a steroid portion, a lactone portion covalently attached to the C-17 of the steroid and a glycoside portion, covalently attached to the C-3 of the steroid portion via a glycosidic linkage. The steroid portion and the lactone portion form the aglycone steroid nucleus of the cardiac glycosides. Some cardiac glycoside are aglycones without the glycoside portion. Two classes of cardiac glycosides are known that are identified by their lactone portion in the aglycone. Cardenolides have an unsaturated butyrolactone ring as lactone portion and bufadienolides have an α-pyrone ring as lactone portion.

Cardiac glycosides are Na⁺/K⁺ ATPase inhibitors that bind to the extracellular part of the phosphorylated Na⁺/K⁺ ATPase that binds potassium to transfer it inside the cell. Extracellular potassium, which induces the dephosphorylation of the alpha subunit of Na⁺/K⁺ ATPase, reduces the effects of cardiac glycosides. Inhibition of Na⁺/K⁺ ATPase results in an intracellular increase of Na⁺. The Na⁺/Ca²⁺ exchanger, which pumps calcium out of the cell and sodium into the cell down their concentration gradient. The decrease in the concentration gradient of sodium into the cell reduces the ability of the Na⁺/Ca²⁺ exchanger to function, resulting in an increase of intracellular calcium levels. In the heart, this results in higher contractility of the cardiac muscle and an increase in the cardiac vagal tone. Cardiac glycosides exert characteristic positively inotropic effects on the heart (increases the strength of cardiac muscle contraction).

In certain embodiments, the cardiac glycoside is selected from a cardenolide and a bufadienolide.

In certain embodiments, the cardiac glycoside is selected from digitoxin, ouabain, convallatoxin, proscillaridin, lanatoside C, gitoformate, peruvoside, strophanthidin, metildigoxin, deslanoside, bufalin, digoxin and digoxigenin.

Digitoxin (CAS 71-63-6) is a cardiac glycoside naturally occurring in the leaves of the foxglove plant (digitalis spec). Digitoxin is commonly used in the treatment of congestive heart failure.

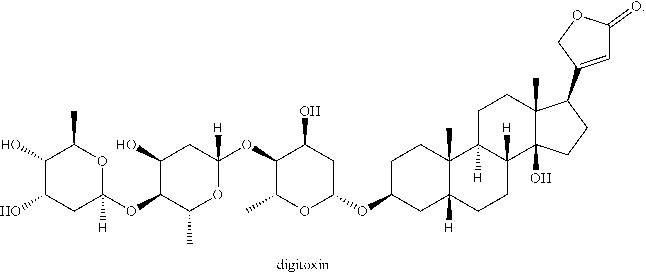

Ouabain (g-strophanthin, (CAS 630-60-4)) is a cardiac glycoside that acts by inhibiting the Na⁺/K⁺-ATPase and is used mainly in the treatment of hypotension and cardiac arrhythmia.

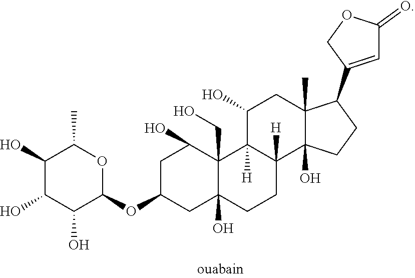

Convallatoxin (CAS 508-75-8) is a cardiac glycoside of the group of the cardenolides and is naturally occurring in convallaria majalis. Convallatoxin has a potency about five times that of digitoxin and is used mainly for the treatment of cardiac arrhythmia.

Proscillaridin (CAS 466-06-8) is a cardiac glycoside of the bufanolide class and is used mainly in the treatment of congestive heart failure and cardiac arrhythmia.

Lanatoside (CAS 17575-22-3) C is a cardiac glycoside of the class of cardenolides and is used mainly in the treatment of congestive heart failure and cardiac arrhythmia.

Gitoformate (CAS 10176-39-3) is a cardiac glycoside of the class of the bufanolide class and is used mainly in the treatment of congestive heart failure and cardiac arrhythmia. Gitoformate is a derivative of the naturally occurring cardiac glycoside gitoxin.

Peruvoside (CAS No. 1182-87-2) is a cardiac glycoside of the class of the bufanolide class and is used mainly in the treatment of congestive heart failure and cardiac arrhythmia.

Strophanthidin is a cardiac glycoside of the class of cardenolides and is used mainly in the treatment of congestive heart failure and cardiac arrhythmia. Strophanthidin is the aglycone of k-strophanthin, which is an analogue of ouabain.

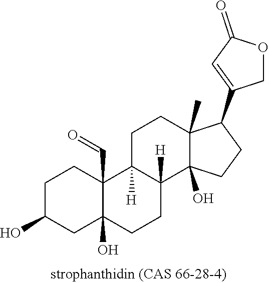

Digoxin is a naturally occurring cardiac glycoside of the class of cardenolides and is used mainly in the treatment of congestive heart failure and cardiac arrhythmia.

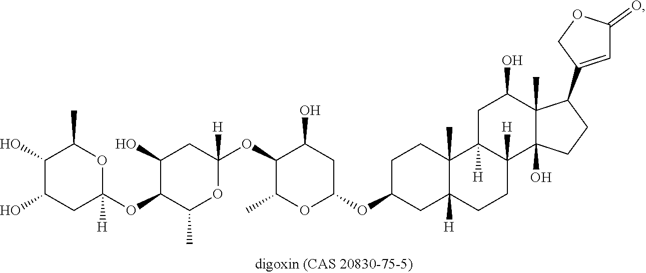

Digoxigenin (CAS 1672-46-4) is a cardiac glycoside of the class of cardenolides. Digoxigenin is the aglycone of digoxin.

Metildigoxin (CAS 30685-43-9) (also referred to as methyldigoxin) is a cardiac glycoside of the class of cardenolides and is used mainly in the treatment of congestive heart failure and cardiac arrhythmia.

Deslanoside (CAS 17598-65-1) is a naturally occurring cardiac glycoside of the class of cardenolides and is used mainly in the treatment of congestive heart failure and cardiac arrhythmia.

Bufalin (CAS 465-21-4) is a naturally occurring cardiac glycoside of the class of bufadienolides.

In certain embodiments, the cardiac glycoside is selected from digoxin, digitoxin and ouabain.

In certain embodiments, the cardiac glycoside is digoxin.

In certain embodiments, the cardiac glycoside is digitoxin.

In certain embodiments, the cardiac glycoside is ouabain.

In certain embodiments, the Na⁺/K⁺ ATPase inhibitor for use in the prevention or treatment of metastasis is for use in the disruption of CTC clusters.

A second aspect of the invention relates to nucleic acid molecule comprising, or consisting of, an inhibitor nucleic acid sequence capable of downregulating or inhibiting expression of a target nucleic acid sequence encoding a protein selected from:
CLDN3,
CLDN4 and
Na+/K+ ATPase or any of its constituent subunit isoforms,

for use in treatment or prevention of metastatic cancer.

The data provided in the examples show that suppression of any of these proteins' expression of function leads to a significant suppression of CTC formation, which in turn is associated with improved clinical outcome in cancer patients.

Claudin 3 (CLDN3; Entrez code 1365) and Claudin 4 (CLDN4; Entrez code 1364) are components of tight junctions and facilitate cell-cell interaction.

In general, both antisense targeting of the gene target implied in formation of CTC clusters and promotion of metastasis, and a CRISPR or analogous approach is contemplated.

In certain embodiments, the inhibitor nucleic acid sequence is able to specifically hybridize with a sequence or subsequence of
an exon comprised in said target nucleic acid sequence,
an intron comprised in said target nucleic acid sequence,
a promoter region modulating expression of said target nucleic acid sequence, and/or
an auxiliary sequence regulating expression of said target nucleic acid sequence.

In certain embodiments, the inhibitor nucleic acid sequence is an antisense oligonucleotide, an siRNA, an shRNA, an sgRNA or an miRNA.

In certain embodiments, the inhibitor nucleic acid sequence comprises or consists of nucleoside analogues.

Hybridization of the inhibitor nucleic acid sequence with the exon, intron, promoter or auxiliary sequence of the target nucleic acid sequence as described above leads to a decreased or inhibited transcription or translation of the target nucleic acid sequence. The mechanism employed may be degradation of mRNA, e.g. by RNA interference, CRISPR/Cas system, inhibition of translation or blockage of a promoter or enhancer region.

In certain embodiments, the auxiliary sequence is an enhancer sequence. The enhancer sequence is a short (50-1500 bp) region of DNA that can be bound by activators to increase the likelihood that transcription of the target nucleic acid sequence will occur. The inhibitor nucleic acid sequence will decrease the activity of the enhancer sequence.

In certain embodiments, the auxiliary sequence is a long non-coding RNA sequence. Long non-coding RNAs are transcripts longer than 200 nucleotides that are not translated into protein, but regulate transcription or translation of the target nucleic acid sequence.

In certain embodiments, said inhibitor nucleic acid sequence is an antisense oligonucleotide. In certain embodiments, said inhibitor nucleic acid sequence is an siRNA. In certain embodiments, said inhibitor nucleic acid sequence is an shRNA. In certain embodiments, said inhibitor nucleic acid sequence is an sgRNA. In certain embodiments, said inhibitor nucleic acid sequence is an miRNA.

In certain embodiments, the inhibitor nucleic acid sequence comprises or consists of nucleoside analogues.

The skilled person is capable of selecting appropriate antisense sequences based on the genetic information contained in public databases on the target sequences.

CTC clusters have a higher potential for metastasis seeding as compared to single circulating tumor cells. Therefore, the ability of the Na⁺/K⁺ ATPase inhibitors and the inhibitor nucleic acid sequence of the present invention to disrupt the CTC clusters into single CTCs is advantageous in the prevention and treatment of cancer patients.

A third aspect of the invention relates to an Na⁺/K⁺ ATPase inhibitor according to the first aspect of the invention or a nucleic acid molecule according to the second aspect of the invention for use in the prevention and treatment of venous thromboembolism in cancer patients.

Presence of CTCs in patients with cancer is associated with an increased risk of venous thromboembolism. Without wishing to be bound by theory this is presumably due to activation of coagulation via CTC-cluster interaction with coagulation or tissue factors in the blood circulation and/or other cell types such as platelets and endothelial cells (Bystricky et al., Critical Reviews in Oncology/Hematology 114: 33-42, 2017).

The Na⁺/K⁺ ATPase inhibitor and the inhibitor nucleic acid sequence of the present invention significantly reduce CTC cluster size and are therefore also able to reduce the incidence of venous thromboembolism in cancer patients.

All embodiments relating to the Na⁺/K⁺ ATPase inhibitor of the first aspect of the invention also relate to the third aspect of the invention.

Another aspect of the invention relates to the use of the Na⁺/K⁺ ATPase inhibitor as characterized above in the manufacture of a medicament for cancer treatment as outlined above. Alternatively, the invention relates to methods for cancer treatment. In such methods, an effective amount of the compound described herein (including a dosage form or formulation as described), is administered to a subject in need thereof, thereby treating the cancer or preventing the spread or recurrence of metastasis.

### Pharmaceutical Compositions and Administration

Another aspect of the invention relates to a pharmaceutical composition comprising a compound of the present invention, or a pharmaceutically acceptable salt thereof, and a pharmaceutically acceptable carrier.

In certain embodiments, the Na⁺/K⁺ ATPase inhibitor according to the invention and any of its aspects and embodiments is formulated as a dosage form for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

In certain embodiments of the invention, the compound of the present invention is typically formulated into pharmaceutical dosage forms to provide an easily controllable dosage of the drug and to give the patient an elegant and easily handleable product.

In embodiments of the invention relating to topical uses of the compounds of the invention, the pharmaceutical composition is formulated in a way that is suitable for topical administration such as aqueous solutions, suspensions, ointments, creams, gels or sprayable formulations, e.g., for delivery by aerosol or the like, comprising the active ingredient together with one or more of solubilizers, stabilizers, tonicity enhancing agents, buffers and preservatives that are known to those skilled in the art.

The pharmaceutical composition can be formulated for oral administration, parenteral administration, or rectal administration. In addition, the pharmaceutical compositions of the present invention can be made up in a solid form (including without limitation capsules, tablets, pills, granules, powders or suppositories), or in a liquid form (including without limitation solutions, suspensions or emulsions).

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

The invention is further illustrated by the following examples and figures, from which further embodiments and advantages can be drawn. These examples are meant to illustrate the invention but not to limit its scope.

### BRIEF DESCRIPTION OF THE FIGURES

FIG. 1 shows DNA-methylation analysis of human single CTCs and CTC cluster A) staining of live CTCs for cell surface expression of EpCAM, HER2, and EGFR (Alexa488- or FITC-conjugated), and counterstained with antibodies against CD45 to identify contaminant leukocytes. B) Principal component analysis mainly separated CTCs based on the patient of origin, with CTC clusters (circles) being more heterogeneous compared to single CTCs (triangle). C) NES score representing enrichment of transcription factor binding sites (TFBSs) in CTC-cluster hypomentylated regions (n=1305) and single CTC hypomethylated regions (n=2042), identified using i-cisTarget. D) Gene ontology (GO) enrichment analysis for 166 genes located at hypomethylated regions in CTC clusters (p=<0.05).

FIG. 2 shows DNA-methylation analysis of mouse xenograft single CTCs and CTC cluster A) NES score representing enrichment of transcription factor binding sites (TFBSs) in CTC-cluster hypomethylated regions (n=909) and single CTC hypomethylated regions (n=521), identified using i-cisTarget. B) Only a very small subset of TFBSs are preferentially hypomethylated in either single CTCs (n=13) or CTC clusters (n=9)

FIG. 3 shows RNA Sequencing analysis of single CTCs and CTC clusters isolated from breast cancer patients A) Network analysis of transcripts identified in the CTC cluster-associated modules B) Gene regulatory network analysis showing transcription factor dependence on TFs SIN3A, OCT4 and CBFB that also display hypomethylated binding sites. C) RNA-sequencing analysis of xenograft-derived CTC clusters showed additionally to those genes found enriched in patient's CTC clusters TFs with significantly hypomethylated binding sites such as SIN3A, NANOG, SOX2, RORA, FOXO1 and BHLHE40. D) Gene ontology (GO) enrichment analysis for genes located at hypomethylated regions in CTC clusters. E) Transcription factor target gene analysis for single CTCs further confirmed the activity of c-MYC, as well as E2F4.

FIG. 4 shows a screen for FDA approved compounds that dissociate CTC clusters. A) Left panels: representative images of steady state “unfiltered” and 40 μM-filtered BR16 cells stained with Hoechst and TMRM. Images are taken with a high-content screening microscope. Right panels: representative images of single and clustered CTCs outline based on nuclei proximity (derived from respective left panel images) as determined using Colombus Image data analysis system. The bar graphs show the mean cluster size (area in μM2) and percentage (%) of viability of unfiltered versus filtered BR16 cells (n=3; NS: not significant; ***p<0.001). B) Top panel: the plot shows mean cluster size of BR16 cells treated with each of the 39 cluster-targeting compounds at 4 different concentrations: 5 μM, 1 μM, 0.5 μM, and 0.1 μM. Cluster-targeting compounds include inhibitors of Na⁺/K⁺ ATPase (n=6), HDAC (n=2), nucleotide biosynthesis (n=5), kinase (n=4), GPCR (n=2), cholesterol biosynthesis (n=1) and nuclear export (n=1) as well as tubulin (n=9) and DNA binding (n=8) compounds and antibiotics (n=1). BR16 cells that were untreated or untreated and 40 μM-filtered are shown as controls for comparison. The average value of two independent measurements is shown. Bottom panel: heatmap showing number of nuclei, average TMRM intensity and % viability for BR16 cells treated with cluster-targeting compounds at the indicated concentrations.

FIG. 5 shows the effect of 17-day in vitro treatment of BR16 and BRx50 cell line with 50 nM, 20 nM, 10 nM, 5 nM and 1 nM concentration of digitoxin, ouabain octahydrate and rigosertib on reducing cluster size, number of nuclei, TMRM intensity and % viability relative to untreated or untreated and further 40 μM filtered cells.

FIG. 6: shows the effect of treatment of CTC-derived cell lines with digitoxin and ouabain. (A) Western blot for CLDN3, CLDN4 and GAPDH on BR16 cells with double knockout (KO) of CLDN3 and CLDN4. KO=Knockout. (B) Plot showing the reduction in mean cluster size (area in μm2) of the CLDN3/4 double KO BR16 cells, relative to control BR16 cells. *P<0.05; **P<0.01 by Student's t test. Error bars represent S.E.M.

FIG. 7 Treatment with Na+/K+ ATPase inhibitors suppresses spontaneous metastasis formation; (A) Schematic representation of the experiment; (B) The plots show the total bioluminescence flux at day 0 (left) and day 1 (right) upon tail vein injection of BR16 cells pre-treated with 20 nM digitoxin or ouabain. n=5; *P<0.05 by Student's t test; NS=not significant. Error bars represent S.E.M. (C) Metastasis growth curve over 72 days upon tail vein injection of BR16 cells pre-treated with 20 nM digitoxin or ouabain. n=5; *P<0.05; **P<0.01 by Student's t test; NS=not significant. Error bars represent S.E.M. (D) Schematic representation of the experiment. (E) The plots show the percent (%) of spontaneously-generated single CTCs and CTC clusters detected in the blood of BR16 xenografts treated with ouabain. n=11 for controls, n=5 for ouabain; ***P<0.001 by Student's t test; Error bars represent S.E.M. (F) The plot shows the metastatic index of BR16 xenografts treated with ouabain. n=11 for controls, n=5 for ouabain; **P<0.01 by Student's t test; NS=not significant. Error bars represent S.E.M. (G) Representative images of the bioluminescence signal measured in brain and liver of control and ouabain-treated NSG mice.

FIG. 8 Treatment with digitoxin and ouabain reduces metastasis formation (A) The plots show the percent of Ki67-positive cancer cells detected in the lungs of NSG mice at Day 0 (left) or Day 1 (right) upon injection with BR16 CTC-derived cells, treated in vitro with digitoxin or ouabain. Cancer cells are identified through Pan Cytokeratin staining; n=4 mice for each condition. Error bars represent S.E.M.; NS=Not significant. (B) The plots show the percent of Caspase 3-positive cancer cells detected in the lungs of NSG mice at Day 0 (left) or Day 1 (right) upon injection with BR16 CTC-derived cells, treated in vitro with digitoxin or ouabain. Cancer cells are identified through Pan Cytokeratin staining; n=4 mice for each condition. *P<0.05 by Student's t test; Error bars represent S.E.M.; NS=Not significant. (C) The plots show the total bioluminescence flux emitted from the primary tumour of BR16 xenografts treated with vehicle (control) or ouabain. Error bars represent S.E.M.; NS=not significant. (D) The plots show the total number of CTCs, including both single CTCs and CTC clusters, detected per mL of blood in BR16 xenografts treated with vehicle (control) or ouabain. n=5 for controls and n=5 ouabain; Error bars represent S.E.M.; NS=Not Significant. (E) The plots show the percent (%) of spontaneously-generated single CTCs and CTC clusters detected in the blood of LM2 xenografts treated with vehicle (control) or ouabain. n=11 for controls, n=8 for ouabain; **P<0.01. (F) The plots show the total bioluminescence flux emitted from the primary tumour of LM2 xenografts treated with vehicle (control) or ouabain. Error bars represent S.E.M.; NS=not significant. (G) The plots show the total number of CTCs, including both single CTCs and CTC clusters, detected per mL of blood in LM2 xenografts treated with vehicle (control) or ouabain. n=11 for controls and n=8 ouabain; Error bars represent S.E.M.; NS=Not significant. (H) The plot shows the metastatic index of LM2 xenografts treated with vehicle (control) or ouabain. n=19 for controls, n=8 for oubain. **P<0.01 by Student's t test; Error bars represent S.E.M.

FIG. 9 shows data derived with the same methodology as the data of FIG. 4 b.

FIG. 10 The plot shows tumor growth rate over time in BR16 xenografts, treated with vehicle (control) or digoxin (2 mg/kg). No significant differences are observed (P>0.05 for all).

FIG. 11 The plot shows the number of single CTCs, CTC clusters and CTC-neutrophil clusters (represented as single CTC-WBC and CTC cluster-WBC) in BR16 xenografts, treated with vehicle (control) or digoxin (2 mg/kg). Digoxin treatment results in a clear decrease in the number of CTC clusters and CTC-neutrophil clusters.

FIG. 12 Plot showing the metastatic index of BR16 xenografts, treated with vehicle (control) or digoxin (2 mg/kg). Treatment with digoxin suppresses metastasis.

FIG. 13 The plot shows tumor growth rate over time in LM2 xenografts, treated with vehicle (control) or digoxin (2 mg/kg). No significant differences are observed (P>0.05 for all).

FIG. 14 Kaplan Meier curve showing overall survival of LM2 xenografts treated with vehicle (control) or digoxin (2 mg/kg). Digoxin treatment prolongs overall survival.

FIG. 15 The plot shows the CTC fold change in LM2 xenografts, treated with vehicle (control) or digoxin (2 mg/kg). Treatment with digoxin reduces the formation of CTC clusters and CTC-neutrophil clusters.

### EXAMPLES

Abnormal DNA methylation patterns, including both genome-wide hypomethylation and hypermethylation have been associated with several human cancers (Klutstein et al., Cancer research 76, 3446-3450, 2016; Ehrlich Epigenomics 1, 239-259, 2009; Ehrlich, M. Oncogene 21, 5400-5413, 2002; Feinberg et al., Nat Rev Genet 7, 21-33, 2006). Generally, these cancer-associated epigenetic modifications appear to affect distinct genomic areas, with hypomethylation favoring regulatory and repetitive elements, versus hypermethylation being more frequent in CpG islands (Ehrlich, M. Oncogene 21, 5400-5413, 2002). Yet, both modifications have the ability to alter the expression of neighboring genes and to contribute to the cancer phenotype (Klutstein et al., Cancer research 76, 3446-3450, 2016; Ehrlich Epigenomics 1, 239-259, 2009). With regard to regulatory elements, loss of DNA methylation at transcription factor binding sites (TFBSs) can designate active transcription factor (TF) networks, or networks that are primed for activation at later stages, e.g. during derivation of induced pluripotent stem cells from differentiated cells (Lee et al., Nat Commun 5, 5619, 2014) or cancer progression. However, the forces that shape the DNA methylome in breast cancer patients and whether distinct DNA methylation patterns dictate the metastatic potential of CTCs is unknown.

DNA-Methylation Pattern of Circulating Tumor Cells (CTC) and CTC Clusters from Breast Cancer Patients

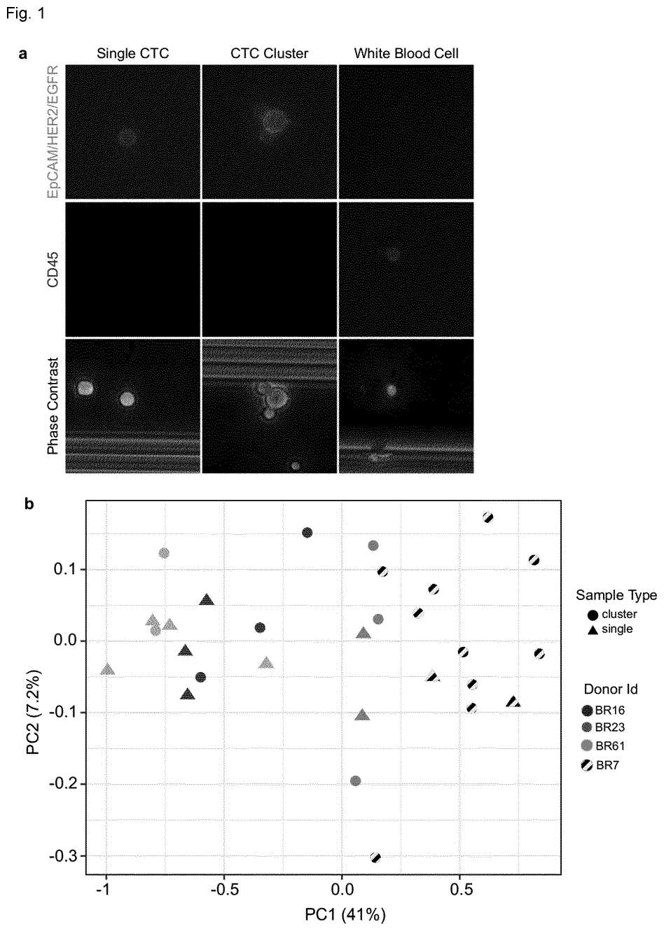

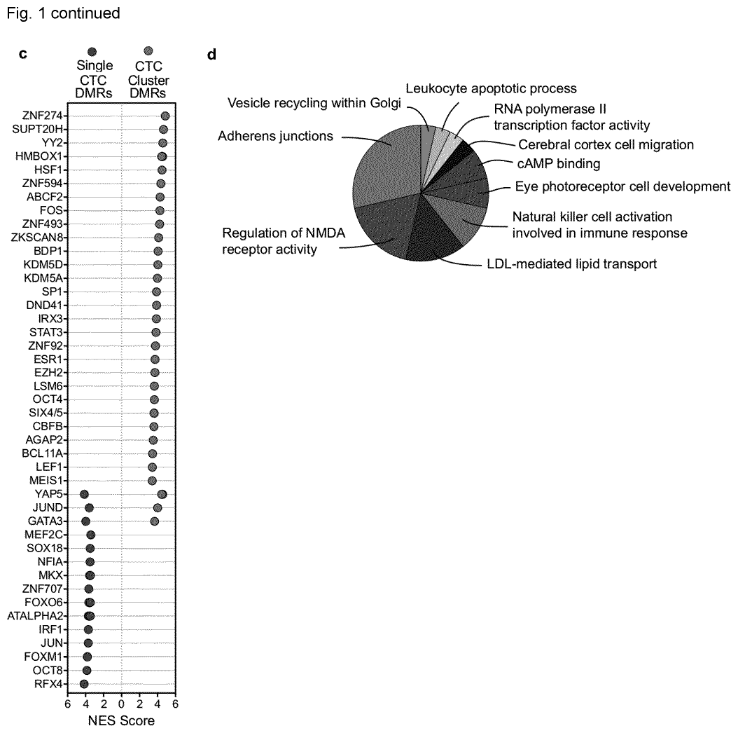

The inventors sought to identify active transcription factor networks by means of accessible TFBSs of single and clustered human breast CTCs, matched within individual liquid biopsies, through a genome-wide single cell-resolution DNA methylation analysis (bisulfite sequencing). To this end, blood samples were drawn from four patients with progressive metastatic breast cancer (Table 1) and processed with Parsortix (Xu et al. PLoS One 10, e0138032, 2015), a microfluidic device that allows a size-based, antigen-agnostic enrichment of CTCs from unmanipulated blood samples. Upon capture, live CTCs were stained for cell surface expression of EpCAM, HER2, and EGFR (Alexa488- or FITC-conjugated), and counterstained with antibodies against CD45 to identify contaminant leukocytes (FIG. 1a). Upon staining verification, a total of 18 marker-positive single CTCs and 24 marker-positive CTC clusters (mean of 5±2.58 single CTCs and 6±4.24 CTC clusters per patient) were then individually micromanipulated (CellCelector) and deposited in lysis buffer for single cell whole-genome bisulfite sequencing (Farlik et al., Cell Rep 10, 1386-1397, 2015; Farlik et al., Cell Stem Cell 19, 808-822, 2016).

TABLE 1
Breast cancer patient information at the time of CTC collection
for WGBS and/or RNA sequencing analysis

| Patient | | | % ER⁺/ | | Metastatic | # of detected |
|---|---|---|---|---|---|---|
| ID | Age | Stage | PR⁺ | HER2 | Sites | CTCs |
| BR7 | 42 | IV | 90/90 | — | Bone | 6 single, |
| | | | | | | 11 clusters |
| BR16 | 49 | IV | 100/75 | — | Bone, Liver, | 8 single, |
| | | | | | Peritoneum, | 3 clusters |
| | | | | | Meningeosis | |
| BR23 | 64 | IV | 100/0 | — | Peritoneum, | 2 single, |
| | | | | | Uterus, | 8 clusters |
| | | | | | Ovaries | 1 single, |
| | | | | | | 4 clusters |
| BR61 | 63 | IV | 60/0 | — | Bone, Soft | 4 single, |
| | | | | | Tissues, | 2 clusters |
| | | | | | Lymphnodes | 9 single, |
| | | | | | | 9 clusters |
| BR11 | 58 | IV | 0/0 | — | Skin, Liver | 11 singles; |
| | | | | | | 2 clusters |
| BR39 | 53 | IV | 60/30 | — | Bone, Liver, | 14 singles, |
| | | | | | Pleura | 7 clusters |
| BR53 | 59 | IV | >1/0 | — | CNS, Lung, | 1 cluster |
| | | | | | Liver, | |
| | | | | | Peritoneum | |
| BR57 | 56 | IV | 95/5 | — | Bone, Liver, | 13 single, |
| | | | | | Lymphnodes | 1 cluster |

Principal component analysis (PCA) mainly separated CTCs based on the patient of origin, with CTC clusters being more heterogeneous compared to single CTCs (FIG. 1b). To identify differentially methylated regions (DMRs) between single CTCs and CTC clusters, methylation in 5 kb windows that are common between at least two different samples in each group was evaluated, and 3′347 DMRs were identified, with a 80% methylation difference between single CTCs and CTC clusters. Of these, 1′305 DMRs were hypomethylated in CTC clusters and 2′042 were hypomethylated in single CTCs. DMRs were analyzed with i-cisTarget, an integrative genomics method that predicts cis regulatory features in co-regulated sequences (Herrmann et al., Nucleic Acids Res 40, e114, 2012). Within CTC cluster hypomethylated DMRs, a significant enrichment for several TFBSs was found, including stemness-related TFs such as OCT4 and STAT3 (FIG. 1c). In contrast, hypomethylated DMRs in single CTCs were enriched in TFBSs for TF such as MEF2C and SOX18 (FIG. 1c). The genomic regions enrichment of annotations tool (GREAT) (McLean et al. Nat Biotechnol 28, 495-501, 2010) was used to identify specific genes that were associated with hypomethylated regions in CTC clusters. Using an association rule of basal plus 50 kb maximum extension, this analysis revealed 166 genes that are associated with gene ontology (GO) categories related to processes that involve cell-cell junction and membrane receptor activity such as adherens junctions, NMDA receptor activity and lipid transport, as well as immune response, including NK cell activation and leukocyte apoptosis (FIG. 1d and Table 2). As a parallel approach, global DNA methylation differences were assessed at TFBSs (Farlik et al., Cell Stem Cell 19, 808-822, 2016) and found OCT4 binding sites to be consistently hypomethylated in CTC clusters (Table 3). Binding sites for other pluripotency-related TFs such as SOX2 and ESRRB were also hypomethylated, as well as binding sites for cell cycle progression-related TFs such as SIN3A (Table 3). In contrast, in single CTCs, with this approach hypomethylation at TFBSs for several TFs were observed including c-MYC and E2F4 (Table 3). Together, the results suggest that CTC clusters display an accessible stemness-related OCT4-centric TF network as well as a cell cycle progression-related SIN3A-centric TF network, paralleling embryonic stem cells (ESCs) biology, whereby these networks simultaneously regulate self-renewal and proliferation (Niwa, Development 134, 635-646, 2007; Kim et al., Cell 132, 1049-1061, 2008; van den Berg et al., Cell Stem Cell 6, 369-381, 2010). Differently, single CTCs appear to be characterized by a c-MYC-centric network, which is commonly enriched in various cancers, yet largely independent of a core pluripotency network and more involved in the regulation of genes associated with metabolism (Kim et al., Cell 132, 1049-1061, 2008; Kim et al. Cell 143, 313-324, 2010).

TABLE 2
Genes identified by GREAT as associated with CTC cluster
hypomethylated DMRs in breast cancer patients.

| | | | |
|---|---|---|---|
| ACER3 | ENSG00000261833 | MBTPS2 | PVRL3 |
| ADAMTS18 | ENSG00000269964 | MEF2C | RAB3C |
| ALG10 | EPS8 | MLIP | RELN |
| ALG6 | EYA4 | MMP26 | RERG |
| AMBN | FAM174A | MRAP2 | RGPD4 |
| ANGPT1 | FAM98B | MSC | RGS7BP |
| ANGPTL1 | FERMT1 | MUC7 | RHOJ |
| ANO3 | FRG2C | MYH4 | RHOXF2B |
| AR | GALNTL6 | MYH8 | RORB |
| ARHGAP6 | GAP43 | NAALADL2 | SATB1 |
| ASXL3 | GCC1 | NALCN | SDIM1 |
| B3GAT2 | GOLGA8B | NAP1L6 | SKAP2 |
| BTNL3 | GOLGA8H | NCAM1 | SLCO1B1 |
| C10orf25 | GOLGA8Q | NEGRI | SLIT2 |
| C10orf35 | GPR85 | NEUROG3 | SNX19 |
| C4orf3 | GRXCR1 | NLGN1 | SPANXC |
| C4orf40 | GUCY2F | NME8 | SPATA22 |
| CA10 | HCN1 | NPC2 | STARD4 |
| CAPN6 | HNRNPA1L2 | NPR3 | STXBP5L |
| CCDC39 | HYDIN | NR2F2 | SYNDIG1L |
| CCDC66 | IFNA14 | NRXN3 | TMEM64 |
| CDH6 | IFNA5 | NUDT10 | TMPRSS11F |
| CDH9 | IFNG | NXT2 | TPH2 |
| CENPE | INSIG2 | OPRM1 | TRIM49D1 |
| CFHR3 | INSL6 | OR13H1 | TRIM64B |
| CHCHD4 | IRS2 | OR1E2 | TRPC6 |
| CLEC2A | ITGBL1 | OR4D5 | TSKU |
| CLSTN3 | ITM2A | OR4M1 | UBE2E2 |
| CNTNAP2 | JRKL | OR4N2 | UGT2A2 |
| CNTNAP5 | KCNIP4 | OR4P4 | UGT8 |
| COL11A1 | KCTD8 | OR8D4 | UPK1B |
| COL12A1 | KL | PABPC5 | USP53 |
| CRB1 | KLHL1 | PDE10A | VAMP7 |
| CTAG1B | KLRF2 | PDE4B | VGLL2 |
| DACH2 | LCA5 | PDHA2 | XXYLT1 |
| DGKH | LDOC1 | PEX5 | ZBTB41 |
| DHRS4L2 | LGALS14 | PLXDC2 | ZKSCAN7 |
| DHRS7 | LGALS16 | POU3F4 | ZNF208 |
| DNAH6 | LPAR4 | PPP1R3A | ZNF676 |
| ENAM | LRRC16B | PRL | ZNF729 |
| ENSG00000176134 | LUM | PRSS37 | |
| ENSG00000257062 | MAX | PTPRZ1 | |

Association rule: Basal + extension: 5000 bp upstream, 1000 bp downstream, 50000 bp max extension, curated regulatory domains included.

TABLE 3
Global methylation differences at TFBS
in single CTCs versus CTC clusters

| Hypomethylated in CTC cluster | | Hypomethylated in single CTC |
|---|---|---|
| SOX2 | SP2 | POL2 |
| STAT1 | BCL3 | TLX1 |
| CTCF | YY1 | c-MYC |
| BRCA1 | MAFF | POL2 |
| ESRRB | RAD21 | KAP1 |
| MAFK | TAL1 | PGC1A |
| CTCF | EGR-1 | MBD4 |
| YY1 | TR4 | RUNX1 |
| EZH2 | NFKB | IRF3 |
| STAT1 | EZH2 | MYB |
| POL2 | OCT4 | JUND |
| MYB | NFKB | IRF3 |
| SIN3A | LYL1 | DDIT3 |
| BCLAF1 | | POL2 |
| ZBTB33 | | NFKB |
| POL2 | | BRF2 |
| ATF3 | | E2F4 |
| NR2F2 | | PAX5 |
| PML | | POL2 |
| p300 | | ZNF |
| RAD21 | | CTCF |

DNA-Methylation Pattern of Circulating Tumor Cells (CTC) and CTC Clusters from an Established Mouse Model

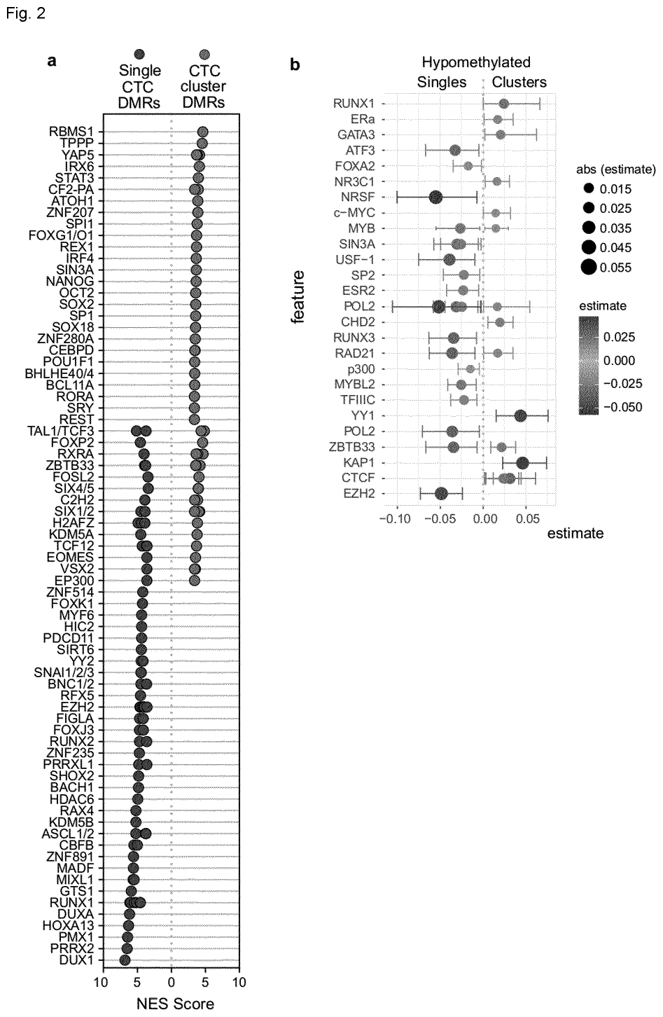

Spontaneously-generated GFP-labeled single CTCs and CTC clusters from three independent mouse xenograft models, including two human breast CTC-derived cell lines (BR16 and BRx50) as well as the breast cancer cell line MDA-MB 231 (lung metastatic variant, referred to as LM2) (Yu et al., Science 345, 216-220, 2014; Minn et al. Nature 436, 518-524, 2005), were isolated to test the robustness of the findings. In this setting, 71 single CTCs and 47 CTC clusters (Table 4) were individually micromanipulated and processed for single cell whole-genome bisulfite sequencing (Farlik et al., Cell Rep 10, 1386-1397, 2015; Farlik et al., Cell Stem Cell 19, 808-822, 2016). Similarly, to patient's CTCs, PCA analysis of xenograft CTCs segregated them primarily based on the cell line of origin, yet displaying an overall higher homogeneity of the samples compared to patient's CTCs. DMRs with a >70% methylation difference between single CTCs and CTC clusters were assessed and a total of 1,430 DMRs were found, of which 909 are hypomethylated in CTC clusters and 521 are hypomethylated in single CTCs. Using i-cisTarget analysis, 40 TFBSs were identified that were hypomethylated in CTC clusters, and 74 TFBSs that were hypomethylated in single CTCs (FIG. 2a). Interestingly, in line with patient's data, both the binding sites for the OCT4-centric TF network, such as those belonging to SOX2, NANOG, STAT3 and REX1, and that of SIN3A were hypomethylated in xenograft CTC clusters. In contrast to patient CTCs though, the sternness-related TF network accessibility seemed to be regulated by localized DNA methylation remodeling at DMRs rather than affecting the global DNA methylation profile of CTCs. This was corroborated by the finding that only a handful of TFBSs are preferentially hypomethylated in either single CTCs (n=13) or CTC clusters (n=9) (FIG. 2b). Thus, distinct DNA methylation profiles of patient and xenograft CTCs seem to reflect their clustering status. It also indicates that, in breast cancer, interplay between methylation dynamics and phenotypic properties of CTCs occurs, and that CTC clustering is associated to an epigenetic predisposition to undergo stemness-related processes and cell cycle progression.

TABLE 4
Number of single CTCs and CTC clusters
isolated per BR16, BRx50 and LM2
injected xenograft mouse models and
used for WGBS or RNA sequencing analysis.

| Mouse ID | Number of CTCs | WGBS | RNA-Seq |
|---|---|---|---|
| BR16-1 | 10 single; 7 clusters | ✓ | — |
| BR16-2 | 23 single; 19 clusters | ✓ | — |
| BR16-3 | 6 single; 15 clusters | — | ✓ |
| BR16-4 | 16 single, 23 clusters | — | ✓ |
| BR16-5 | 10 singles, 4 clusters | — | ✓ |
| BRX50-1 | 10 single, 3 clusters | ✓ | — |
| BRX50-2 | 2 single, 1 cluster | ✓ | — |
| BRX50-3 | 4 single, 2 clusters | — | ✓ |
| LM2-1 | 16 single; 10 clusters | ✓ | — |
| LM2-2 | 10 single; 7 clusters | ✓ | — |
| LM2-3 | 5 singles; 3 clusters | — | ✓ |
| LM2-4 | 5 singles; 2 clusters | — | ✓ |
| LM2-5 | 3 singles; 5 clusters | — | ✓ |

### Stem-Cell Like Related Transcription Factor Networks

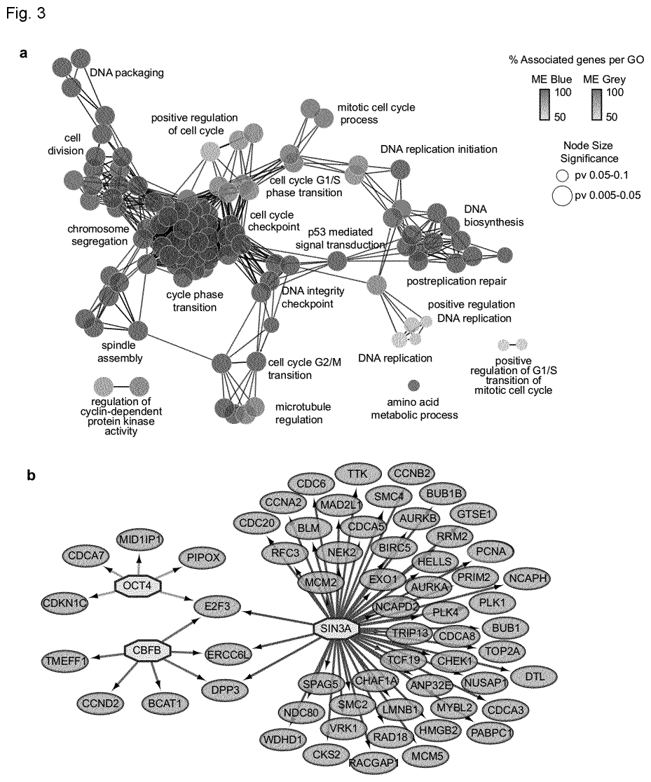

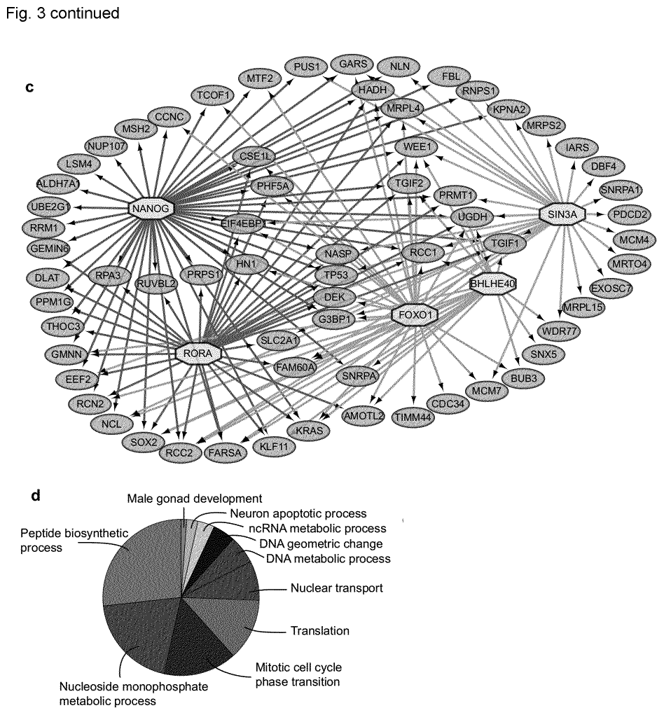

To identify whether the accessible stemness-related TFs networks are also transcriptionally active, the inventors performed single cell-resolution RNA-Sequencing analysis of 48 single CTCs and 24 CTC clusters, matched within individual liquid biopsies and isolated from 6 breast cancer patients with progressive metastatic disease, and of 49 single CTCs and 54 CTC clusters isolated from the three xenograft mouse models (Table 4). A set of 335 genes that were previously shown to be consistently upregulated in mouse and human embryonic stem cells and embryonal carcinoma cells as opposed to their differentiated counterparts was further investigated (Wong et al. Cell Stem Cell 2, 333-344, 2008). A subset of 301 of these 335 genes were found to be expressed in the CTC samples. With these genes, a weighted gene co-expression network analysis (WGCNA) was performed and four expression modules in human breast cancer samples (blue, grey, turquoise and brown) and four expression modules in xenograft CTC clusters were identified (green, yellow, orange, purple), revealing module-trait relationships in CTCs. Particularly, 85 transcripts enriched in patient CTC clusters and 153 in xenograft CTC clusters were identified (Table 5 and 6) with 90% overlap between patient and xenograft CTC-cluster-enriched stemness-related transcripts. Interestingly, transcripts enriched in patient's CTC clusters, as well as those that overlap between patients and xenografts, are mostly involved in cell cycle progression as judged by their network analysis (FIG. 3a), while TF target gene analysis confirmed, among others, activity of TFs SIN3A, OCT4 and CBFB with significantly hypomethylated binding sites (FIG. 3b). In a similar fashion, in xenograft-derived CTC clusters, additionally to those genes found enriched in patient's CTC clusters, TF target gene analysis highlighted the activity of OCT4 including TFs with significantly hypomethylated binding sites such as SIN3A, NANOG, SOX2, RORA, FOXO1 and BHLHE40 (FIG. 3c). TF target gene analysis for single CTCs further confirmed the activity of c-MYC, as well as p53 and E2F4, among others (FIG. 3e). Together, the gene expression data supports the model proposed with DNA methylation analysis, demonstrating that CTC clusters are primed for an OCT4-centric stemness-related TF network and display activation of a SIN3A-dependent cell cycle progression program. Activation of these programs plays a role in determining the metastasis-seeding ability of CTC clusters.

DNA methylation patterns in CTC clusters shape an accessible and active transcription factor network that gives a proliferation advantage in CTC clusters over single CTCs in breast cancer patients. The forces that shape the DNA methylome involve both global differences at TFBSs as well as localized events that mediate response to environmental cues and phenotypic properties. Harnessing the ability to dynamically shape the DNA methylome in response to environmental stimuli can be exploited therapeutically by repurposing FDA approved compounds.

TABLE 5
Weighted gene co-expression network analysis
(WGCNA) of stemness related genes
in breast cancer patient CTCs and distribution
of genes per expression module
Blue Module

| | | | |
|---|---|---|---|
| BIRC5 | MYBL2 | EXO1 | COQ3 |
| BLM | NEK2 | AURKB | ERCC6L |
| BUB1 | PCNA | TRIP13 | CDCA8 |
| BUB1B | PLK1 | GNA14 | RAD18 |
| CCNA2 | POLD1 | NCAPD2 | LSM2 |
| CCNF | POLE2 | CHAF1A | ANP32E |
| CDC6 | PRIM1 | SMC4 | CDCA3 |
| CDC20 | PRIM2 | NDC80 | CDCA7 |
| CDKN3 | RFC3 | SMC2 | CDCA5 |
| CHEK1 | RPA2 | SPAG5 | |
| CKS2 | RRM2 | PLK4 | |
| DNMT1 | SLC16A1 | WDHD1 | |
| HELLS | AURKA | CHEK2 | |
| HMGB2 | TCF19 | NCAPH | |
| LMNB1 | TOP2A | CKAP2 | |
| MAD2L1 | TTK | RACGAP1 | |
| MCM2 | VRK1 | NUSAP1 | |
| MCM3 | CDC7 | GTSE1 | |
| MCM5 | CCNB2 | DTL | |

Grey Module

| | | | |
|---|---|---|---|
| ALDOC | GLDC | ELOVL6 | TMEFF1 |
| BCAT1 | E2F3 | MID1IP1 | U2AF1 |
| CCND2 | PABPC1 | PIPOX | GSPT2 |
| CDKN1C | ACAD8 | PRMT3 | RAB34 |
| FGFR1 | LSM10 | DPP3 | |

Brown Module

| | | | |
|---|---|---|---|
| SLC25A5 | GLO1 | PSMA7 | EIF2S2 |
| ALDH7A1 | HSPE1 | RPA3 | GNPDA1 |
| ATP5J | IARS | RPL13 | LYPLA1 |
| ATP5O | RPSA | RPL22 | MTHFD2 |
| BTF3 | NAP1L1 | RPL27A | RUVBL2 |
| CKS1B | NDUFA9 | RPS3 | EBNA1BP2 |
| COX5B | NDUFB7 | RPS5 | LSM5 |
| CTNNA1 | NDUFB8 | RPS8 | TIMM13 |
| DTYMK | RPL10A | RPS12 | TIMM8B |
| ECHS1 | NME2 | RPS16 | CYCS |
| EIF4EBP1 | PA2G4 | RPS19 | NT5DC2 |
| ENO1 | PRDX1 | RPS23 | THOC3 |
| ETFA | POLR2F | RPS27 | FAM136A |
| FDPS | PRPS1 | SNRPA | NDUFA11 |

Turqoise Module

| | | | |
|---|---|---|---|
| ABCB7 | NDUFAB1 | ZNF22 | STOML2 |
| ADH5 | NDUFS2 | DAP3 | GMNN |
| PARP1 | NME4 | DEK | MRPL4 |
| ADSL | NONO | CLPP | UTP18 |
| APEX1 | NTHL1 | TEAD2 | MRPS2 |
| BAX | PDCD2 | NIPSNAP1 | MRTO4 |
| CCND1 | PDHA1 | HAT1 | HN1 |
| SERPINH1 | PHB | RUVBL1 | HSPA14 |
| CCNC | PPM1G | BANF1 | MRPL37 |
| CDC34 | PPP4C | PROM1 | MRPS17 |
| CTSC | MAPK13 | DDX18 | NIP7 |
| RCC1 | PSMB5 | BUB3 | AMOTL2 |
| CRABP2 | PSMB6 | EEF1E1 | POLR3K |
| CSE1L | RAD23B | PDIA4 | WBP11 |
| CSRP2 | RCN2 | G3BP1 | MRPL39 |
| DHX9 | RRM1 | PSME3 | MRPL16 |
| TIMM8A | SARS | PSMD14 | DARS2 |
| DLAT | SDHC | POP7 | IPO9 |
| PHC1 | SDHD | YAP1 | RCC2 |
| EEF2 | SET | TIMM44 | NUP107 |
| EIF2S3 | SLC2A1 | RPP40 | NLN |
| EIF4A1 | SNRPA1 | MRPS30 | FAM60A |
| EIF4B | SNRPD1 | RNPS1 | TGIF2 |
| FBL | SQLE | DBF4 | MRPL11 |
| FARSA | SSB | ERP29 | C11orf48 |
| FH | SS18 | STIP1 | WDR77 |
| GARS | TCOF1 | CBX3 | C2orf47 |
| GART | TERF1 | MTF2 | GEMIN6 |
| HADH | TGIF1 | NCBP2 | PUS1 |
| HDAC1 | TP53 | SEPHS2 | TCF7L1 |
| HNRNPA1 | TRIP6 | CCT5 | PHF5A |
| PRMT1 | UBE2G1 | EXOSC7 | MRPS36 |
| HSPA9 | UBE2V2 | KPNA6 | NUDCD2 |
| KLF11 | SUMO1 | KLF4 | |
| KPNA2 | UGDH | LSM4 | |
| KRAS | UQCRH | GNL3 | |
| MCM7 | VBP1 | SNX5 | |
| MSH2 | WEE1 | MRPS28 | |
| MYC | XPO1 | MRPS18B | |
| NASP | XRCC5 | MRPL13 | |
| NCL | YY1 | MRPL15 | |

TABLE 6
WGCNA analysis of stemness related
genes in xenograft mouse model CTCs
and distribution of genes per expression module
Green module

| | | | |
|---|---|---|---|
| ALDOC | MSH2 | TMEFF1 | PIPOX |
| BIRC5 | MYBL2 | CCNB2 | AMOTL2 |
| ALDH7A1 | NASP | EXO1 | GTSE1 |
| BLM | NCL | BUB3 | DTL |
| BUB1 | NEK2 | AURKB | COQ3 |
| BUB1B | NTHL1 | TRIP13 | MRPL39 |
| CCNA2 | PCNA | NCAPD2 | ERCC6L |
| CCNC | PDCD2 | CHAF1A | MRPL16 |
| CCNF | PLK1 | SMC4 | CDCA8 |
| CDC6 | POLD1 | G3BP1 | DARS2 |
| CDC20 | POLE2 | PRMT3 | RCC2 |
| CDC34 | PPM1G | POP7 | RAD18 |
| CDKN3 | PRIM1 | NDC80 | NUP107 |
| RCC1 | PRIM2 | TIMM44 | NLN |
| CHEK1 | PRPS1 | SMC2 | FAM60A |
| CKS1B | RCN2 | SPAG5 | TGIF2 |
| CKS2 | RFC3 | PLK4 | MRPL11 |
| CSE1L | RPA2 | MTHFD2 | WDR77 |
| DLAT | RPA3 | RPP40 | C2orf47 |
| DNMT1 | RRM1 | RUVBL2 | GEMIN6 |
| DTYMK | RRM2 | MRPS30 | ANP32E |
| E2F3 | SDHD | RNPS1 | CDCA3 |
| ECHS1 | SLC2A1 | DBF4 | CDCA7 |
| EIF4EBP1 | SLC16A1 | WDHD1 | THOC3 |
| FBL | SNRPA | CHEK2 | PHF5A |
| FARSA | SNRPA1 | MTF2 | CDCA5 |
| GARS | SOX2 | EXOSC7 | NUDCD2 |
| HADH | AURKA | NCAPH | |
| HELLS | TCF19 | GSPT2 | |
| HMGB2 | TCOF1 | LSM4 | |
| PRMT1 | TGIF1 | CKAP2 | |
| IARS | TOP2A | ACAD8 | |
| KLF11 | TP53 | SNX5 | |
| KPNA2 | TTK | MRPL15 | |
| KRAS | U2AF1 | RACGAP1 | |
| LMNB1 | UBE2G1 | GMNN | |
| MAD2L1 | UGDH | MRPL4 | |
| MCM2 | VBP1 | MRPS2 | |
| MCM3 | VRK1 | MRTO4 | |
| MCM4 | WEE1 | HN1 | |
| MCM5 | DEK | NUSAP1 | |
| MCM7 | CDC7 | MRPL37 | |

Yellow module

| | |
|---|---|
| EEF2 | PUS1 |

Orange module

| | | | |
|---|---|---|---|
| ADSL | ELOVL6 | RAB34 | SARS |
| BCAT1 | ENO1 | RPL10A | TEAD2 |
| CCND1 | GJA1 | RPL13 | TIMM8B |
| CCND2 | GNL3 | RPL27A | TRIP6 |
| CDKN1C | HDAC1 | RPS12 | WBP11 |
| CSRP2 | MID1IP1 | RPS16 | YAP1 |
| CTSC | MYC | RPS19 | ZNF22 |
| EBNA1BP2 | NAP1L1 | RPS3 | |
| EIF2S2 | NIP7 | RPS8 | |
| EIF4A1 | POLR2F | RPSA | |

Purple module

| | | | |
|---|---|---|---|
| ABCB7 | NDUFA9 | SSB | NCBP2 |
| ADH5 | NDUFAB1 | SS18 | SEPHS2 |
| PARP1 | NDUFB7 | TERF1 | CCT5 |
| SLC25A5 | NDUFB8 | UBE2V2 | KPNA6 |
| APEX1 | NDUFS2 | SUMO1 | LSM5 |
| ATP5J | NME2 | UQCRH | KLF4 |
| ATP5O | NME4 | XPO1 | TIMM13 |
| BAX | NONO | XRCC5 | PABPC1 |
| BTF3 | PA2G4 | YY1 | MRPS28 |
| SERPINH1 | PRDX1 | DAP3 | MRPS18B |
| COX5B | PDHA1 | CLPP | MRPL13 |
| CRABP2 | PHB | NIPSNAP1 | STOML2 |
| CTNNA1 | PPP4C | HAT1 | UTP18 |
| DHX9 | MAPK13 | RUVBL1 | HSPA14 |
| TIMM8A | PSMA5 | BANF1 | MRPS17 |
| PHC1 | PSMA7 | DDX18 | POLR3K |
| EIF2S3 | PSMB5 | EEF1E1 | CYCS |
| EIF4B | PSMB6 | PDIA4 | IPO9 |
| ETFA | RAD23B | GNA14 | LSM2 |
| FDPS | RPL22 | GNPDA1 | NT5DC2 |
| FGFR1 | RPS5 | DPP3 | C11orf48 |
| FH | RPS23 | PSME3 | TCF7L1 |
| GART | RPS27 | PSMD14 | FAM136A |
| GLO1 | SDHC | LYPLA1 | LSM10 |
| HNRNPA1 | SET | ERP29 | MRPS36 |
| HSPA9 | SNRPD1 | STIP1 | NDUFA11 |
| HSPE1 | SQLE | CBX3 | |

### CTC Cluster Dissociation

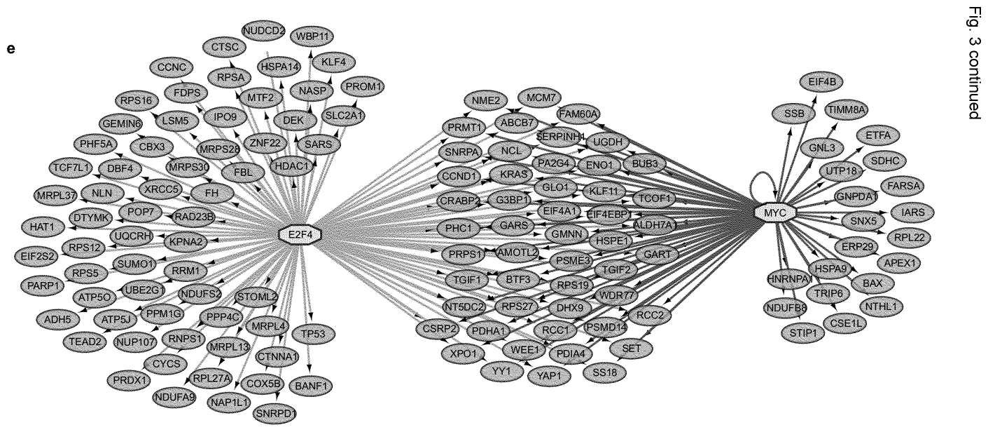

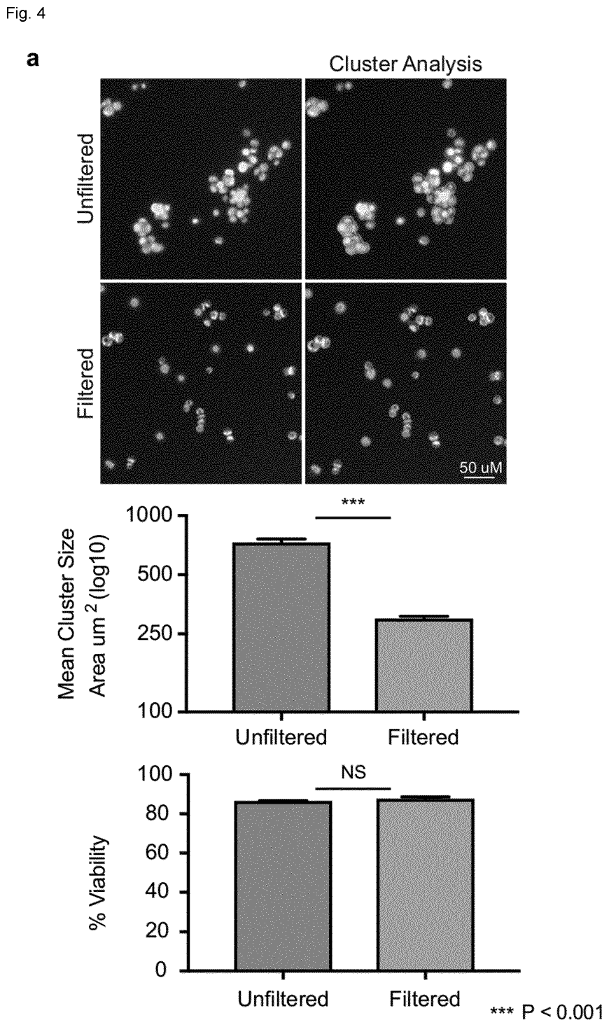

In order to identify actionable vulnerabilities of CTC clusters, and to test whether the epigenetic and transcriptional features of clustered CTCs are reversible upon cluster dissociation into single cells the following steps were undertaken. First, the expression of all known cell-cell junction (CCJ) components in patient samples obtained from normal breast (TGCA REF), breast cancer (TOGA REF), single CTCs and CTC clusters were assessed (Aceto et al. Cell 2014). While breast cancer cells tend to only partially reduce their CCJ repertoire compared to normal breast cells, CTCs express only a small fraction of CCJ components, likely as a consequence to their increased motility. Yet, CTC clusters retain a higher number of CCJs as compared to single CTCs. This analysis features a therapeutic opportunity, and demonstrates that CTC clusters rely upon a restricted number of CCJ components for their multicellular adhesion, with approaches aiming at dissociating them being able to spare normal tissues that express a higher variety of CCJs. To this end, 2′486 FDA-approved compounds were evaluated for their ability to dissociate clusters of human breast CTC-derived cells. Cluster dissociation was assessed using a high content screening microscope and comparing cells treated with each individual compound to steady state clustered BR16 cells and 40 μm-filtered BR16 single cell suspension as negative and positive controls, respectively (FIG. 4a). Interestingly, significant reduction in mean cluster size upon filtration did not affect viability but reduced mitochondrial membrane potential, as measured by tetramethylrhodamine methyl ester perchlorate (TMRM) intensity (FIG. 4a). For the majority of the 2′486 FDA-approved compounds, when using a 5 μM concentration for 2 days in hypoxic conditions, no detectable reduction in cell viability (>70% viability) nor mean cluster size (>450 μm2) in BR16 CTC-derived cells was observed. Yet, 39 compounds were identified that significantly reduced mean cluster size without compromising viability. These compounds include inhibitors of Na⁺/K⁺ ATPases (n=6), HDACs (n=2), nucleotide biosynthesis (n=5), kinases (n=4), GPCRs (n=2), cholesterol biosynthesis (n=1), nuclear export (n=1), tubulin (n=9), as well as DNA binding compounds (n=8) and antibiotics (n=1). Reducing compound concentration to 1 μM, 0.5 μM and 0.1 μM resulted in a concomitant increase in mean cluster size of BR16 as well as BRx50 human CTC-derived cells (FIG. 4b). Along with the effects on cluster size, with a reduced compound concentration, an increase in the number of nuclei detected, mitochondrial membrane potential and overall viability for both cell lines was observed, indicating that cluster size correlates with overall fitness and proliferative ability of CTCs (FIG. 4b). Under these conditions, six compounds consistently led to a significant decrease in mean cluster size for both BR16 and Brx50 CTC cell lines, even at lowest concentrations tested (0.1 μM), namely the Na⁺/K⁺ ATPase inhibitors digitoxin and ouabain octahydrate, the tubulin binding agent podofilox (also known as podophyllotoxin), colchicine and vincristine sulfate, and the tubulin binding agent and dual kinase inhibitor rigosertib (FIG. 4b).

### Effect of CTC Cluster Dissociation on DNA Methylation

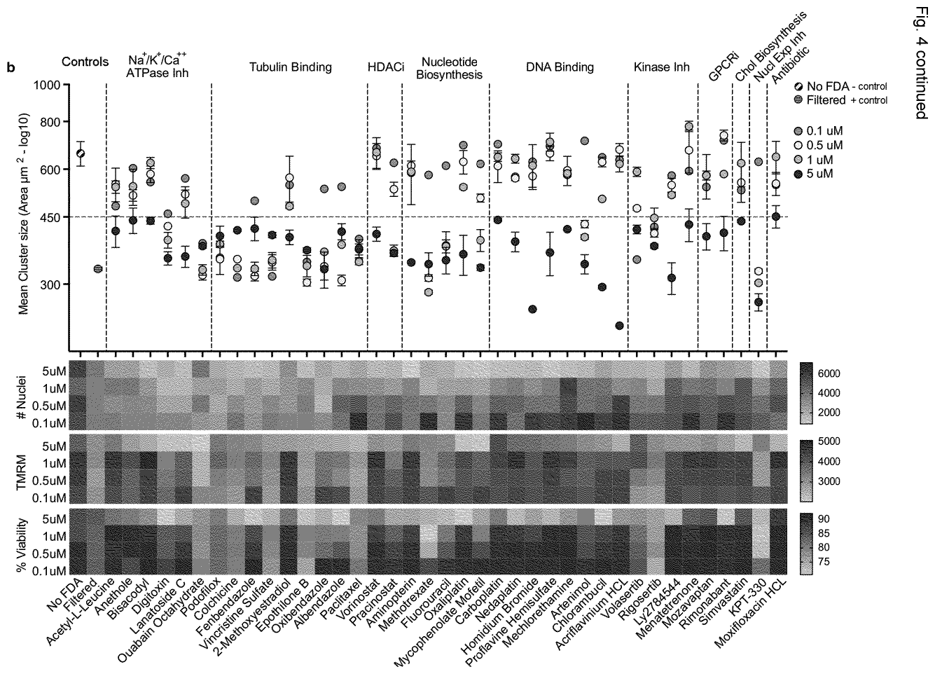

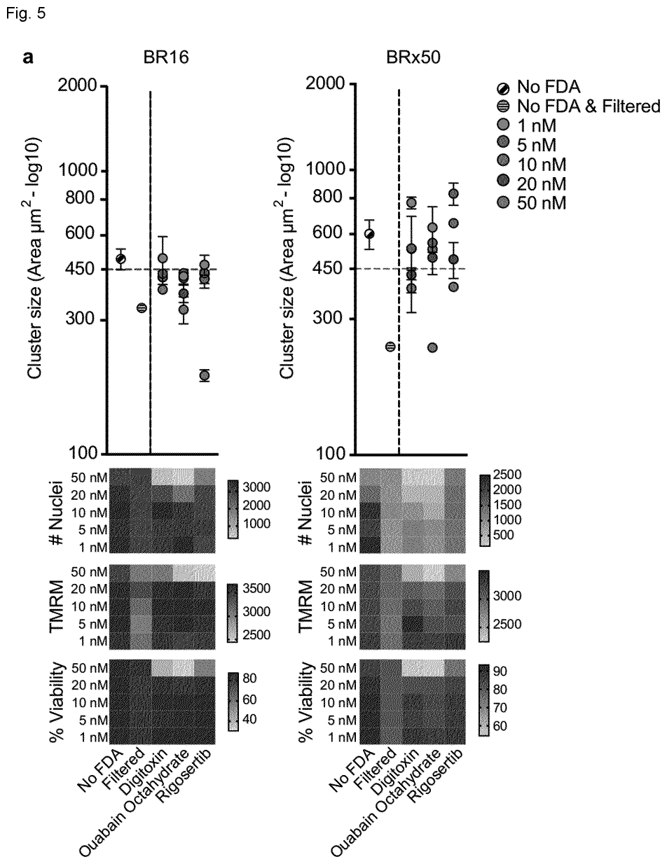

In order to assess the effect of clustering on DNA methylation patterns and proliferation signature directly BR16 and BRx50 cell lines were cultured for 17 days to ensure that at least 4 divisions would take place. This is to allow proper time for DNA methylation remodeling to take place. Prolonged culture in the presence of 20 nM for the ATPase and kinase inhibitors was found to be optimal for cell proliferation and mean cluster size reduction (FIG. 5a) and on average n=20 cells in triplicate were further processed for WGBS and RNA-Seq.

Under these conditions, for both CTC derived cell lines, a subset of cluster-associated hypomethylated DMRs from patient and xenografts regain ≥20% methylation. Interestingly, this gain of methylation occurs in DMRs containing binding sites for stemness-related TFs, with ouabain treatment of BR16 cell line simultaneously affecting the binding sites of OCT4, SOX2 and NANOG. This indicates that the dissociation of CTC-clusters in patient-derived CTC lines leads to DNA remodeling that reduces the accessibility of binding sites for stemness-related TFs.

TABLE 7
NES score of TFBS identified in DMRs with ≥20%
gain in methylation upon 17-day treatment of BR16 and
BRx50 CTC cell line with 20 nM of Ouabain or Digitoxin.

| | BR16 | | BRx50 | |
|---|---|---|---|---|
| | Digitoxin | Ouabain | Digitoxin | Ouabain |
| | (n = 150) | n = (108) | (n = 124) | (n = 121) |
| ZNF274 | — | 4.59, 3.01 | — | — |
| SUPT20H | — | — | — | — |
| YY2 | — | 6.5, | — | 3.56 |
| HMBOX1 | — | 3.89 | 3.749 | 3.35, 3.30, |
| | | | | 3.22 |
| HSF1 | — | 3.71 | 7.2 | — |
| ZNF594 | — | 4.84 | — | — |
| ABCF2 | — | — | — | — |
| FOS | 6.40, 3.63, | 7.29, 6.04, | 4.2, 3.4 | 3.91 |
| | 3.25 | | | |
| ZNF493 | — | — | 3.88 | 3.12 |
| ZKSCAN8 | — | — | 4.32 | — |
| BDP1 | 4.18, | 5.64 | 6.83 | — |
| KDM5D | — | 3.11 | 3.77 | — |
| KDM5A | 4.355 | — | — | — |
| SP1 | — | — | — | — |
| DND1 | — | — | — | — |
| IRX3 | 5 | 3.63 | — | — |
| STAT3 | 3.31 | — | 3.22 | 9.17, 3.80 |
| ZNF92 | 3.44 | — | — | — |
| ESR1 | — | — | — | — |
| EZH2 | 4.63 | — | — | — |
| LSM6 | — | — | 3.05 | — |
| OCT4 | — | 3.2, 3.13 | 3.49, 3.40, | 3.42 |
| | | | 3.37 | |
| SIX4/5 | — | 6.27, 6.16 | 7.50, 7.47, | 3.87, 3.65 |
| | | | 3.12 | |
| CBFB | — | — | — | — |
| AGAP2 | — | — | — | — |
| BCL11A | — | — | 7.65 | 3.8, 3.69 |
| LEF1 | — | — | 3.01 | — |
| MEIS1 | 3.11 | 3 | 6.58, 3.02 | 4.09 |
| RBMS1 | — | — | — | — |
| TPPP | — | 3.1 | — | — |
| YAP5 | 5.4, 5.32, | 3.55, 3.08 | — | — |
| | 4.56 | | | |
| IRX6 | 5, 3.67 | 3.63 | — | 3.8 |
| CF2-PA | — | — | — | — |
| ATOH1 | — | — | — | — |
| ZNF207 | — | — | — | — |
| SPI1 | — | — | — | — |
| FOXG1/O1 | 3.61 | 3.74, 3.39 | — | 3.53, 3.25, |
| | | | | 3.1 |
| REX1 | — | — | — | — |
| IRF4 | — | 3.244 | — | — |
| SIN3A | — | — | — | — |
| NANOG | — | 3.349, 3.28, | — | 3.61 |
| OCT4 | — | 3.204, 3.13 | 3.49, 3.4, | 3.42 |
| | | | 3.37 | |
| SOX2 | 3.22 | 4.82, 4.51, | — | — |
| | | 4.24, 3.28 | | |
| SR1 | — | — | — | — |
| SOX18 | 3.99, 3.91 | 3.2 | — | 3.11 |
| ZNF280A | — | — | — | — |
| CEBPD | 3.76 | 4.05 | — | — |
| POU1F1 | — | — | 4.02, 3.98 | — |
| BHLHE40/4 | — | — | 4.61 | — |
| BCL11A | — | — | 7.65 | 3.8, 3.69 |
| RORA | 4.93, 3.45 | — | — | 4.02, 3.75 |
| SRY | — | 6.46, 6.36, | — | — |
| REST | — | — | 4.85, 3.16, | — |
| | | | 3.11 | |

n = number of DMRs

### CLDN3/CLDN4 Knockout

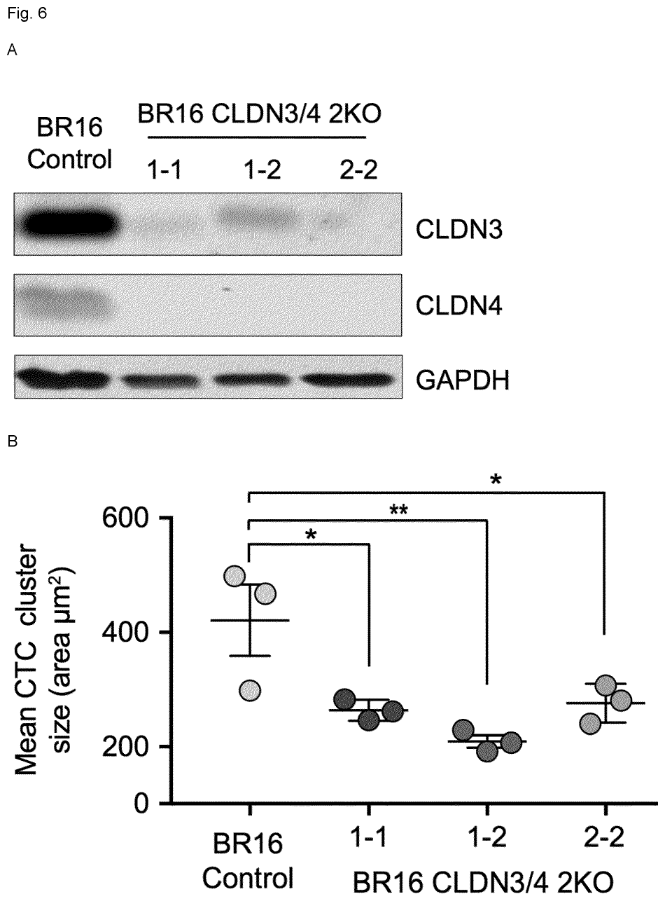

The inventors assessed whether cell-cell junction disruption in CTC-derived cells would lead to clusters dissociation as well as DNA methylation remodeling at CTC cluster-associated DMRs. To this end, the inventors employed the CRISPR technology to simultaneously knockout both claudin 3 (CLDN3) and claudin 4 (CLDN4) in BR16 CTC-derived cells, two of the highest-expressed tight junction proteins in CTC clusters. Using two independent sgRNAs for each gene, the inventors generated three BR16 lines with double CLDN3/4 knockout, which also displayed a significant reduction of mean CTC cluster size (FIG. 6A, 6B).

Whole genome bisulfite sequencing of the CLDN3/4 double knockout cells showed that, upon dissociation into single cells and similarly to the events that occurred upon Na⁺/K⁺ ATPase inhibition, a number of CTC cluster-associated hypomethylated regions gained methylation. Interestingly, i-cis Target analysis of the regions that gained higher levels of methylation revealed an enrichment of binding sites for OCT4, SOX2, NANOG and SIN3A, further indicating that CTC clustering directly impacts DNA methylation dynamics at bindings sites for sternness and proliferation-associated TFs.

Together, the results indicate that Na⁺/K⁺ ATPase inhibition leads to CTC clusters dissociation through the increase of the intracellular Ca++ concentration and the consequent inhibition of cell-cell junction formation, resulting into DNA methylation remodeling at critical stemness- and proliferation-related binding sites.

Treatment with Na+/K+-ATPase Inhibitors Suppresses Spontaneous Metastasis Formation

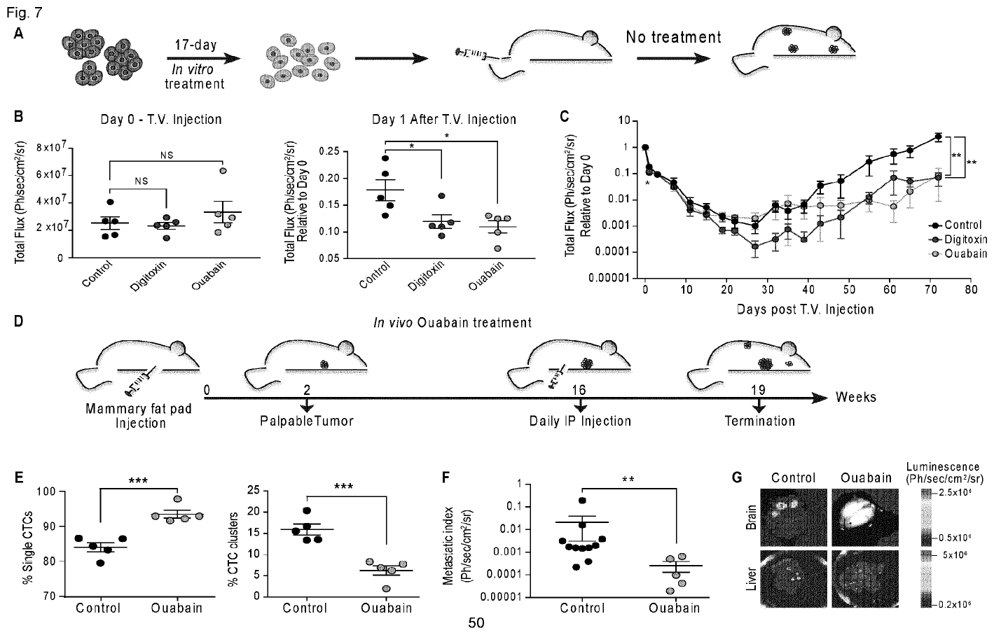

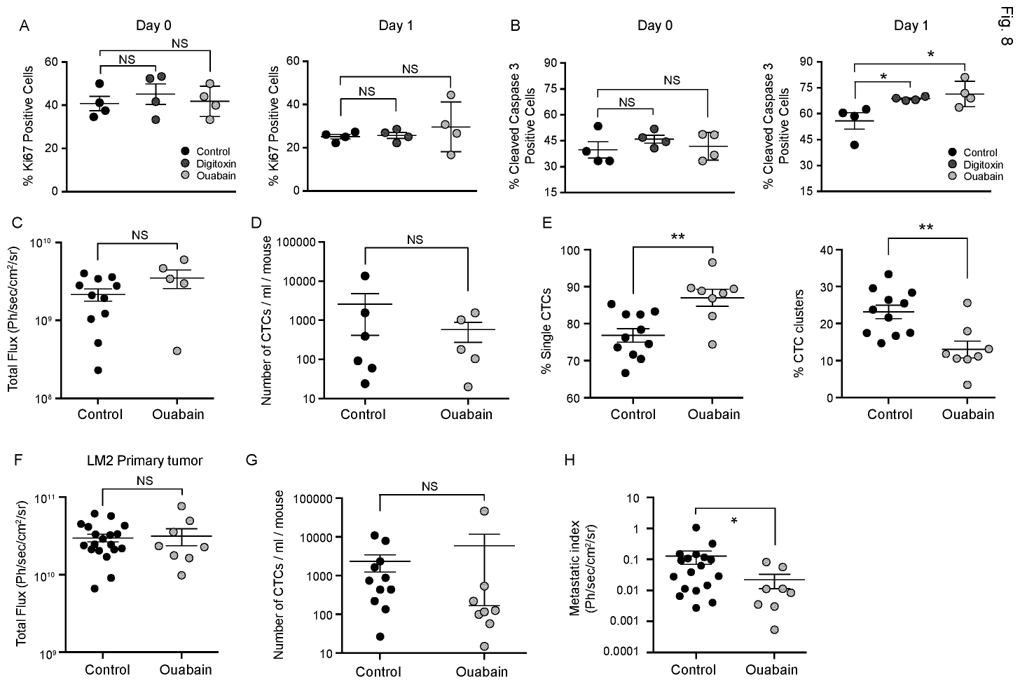

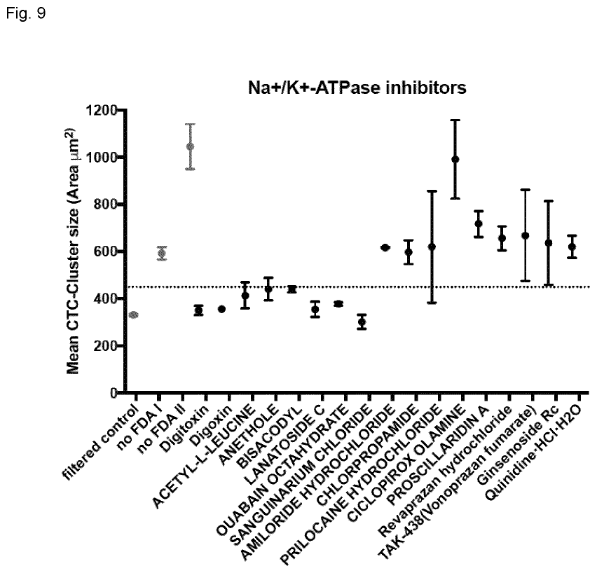

To test whether ouabain and digitoxin would also enable CTC clusters disruption in vivo, the inventors took a dual approach. First, the inventors tested whether a 17-day in vitro treatment with ouabain and digitoxin would translate into a reduced ability of the treated cells to efficiently seed metastasis in untreated mice (FIG. 7A). To this end, upon treatment, BR16 cells stably expressing GFP-luciferase were injected into the tail vein of NSG mice and noninvasively monitored through luminescence imaging for their ability to seed and propagate metastatic lesions. The inventors found that while the treatment with digitoxin or ouabain did not affect the ability of BR16 cells to lodge in the lung tissue immediately after injection (see “day 0”; FIG. 7B), it led to a reduced ability to survive during the first day upon arrival, as confirmed by a significant increase in the expression of cleaved caspase 3 compared to control cells (see “day 1”; FIGS. 7B and 8A, B). Overall, this difference in the ability to survive during the very early steps of metastasis seeding resulted in a delayed metastatic outgrowth despite the absence of further treatment in vivo, as measured during the course of 72 days upon injection (FIG. 7C).

Secondly, mimicking more closely the clinical setting, to assess the effect of our CTC cluster-dissociation strategy for the spontaneous formation of CTC clusters and metastasis from a primary tumor, the inventors injected BR16 cells in the mammary fat pad of NSG mice. 14 weeks after primary tumor formation the inventors administered ouabain daily for three weeks and assessed CTC composition and the occurrence of spontaneous metastatic lesions (FIG. 7D). Importantly, the inventors observed that ouabain treatment reduced the frequency of spontaneously-generated CTC clusters while increasing the frequency of single CTCs (FIG. 7E), without altering the size of the primary tumor nor overall CTC numbers (FIG. 8C, D). Along with a reduction in the frequency of CTC clusters, ouabain treatment also resulted in a remarkable suppression (80.7-fold) of the total metastatic burden (FIG. 7F, G). In a similar fashion, when administering ouabain treatment to NSG mice carrying spontaneously-metastasizing LM2 tumors, the inventors also observed an increase in the proportion of single CTCs and a decrease in CTC clusters (FIG. 8E), without any change in the primary tumor size nor overall CTC numbers (FIG. 8F, G), leading to a reduced metastatic burden compared to control (FIG. 8H).

### Digoxin Treatment

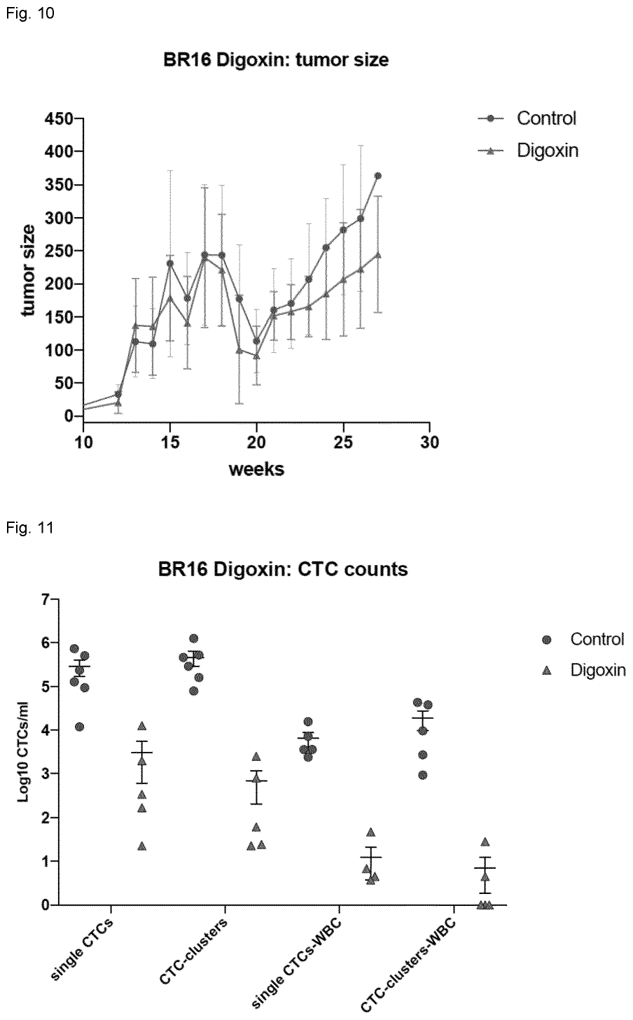

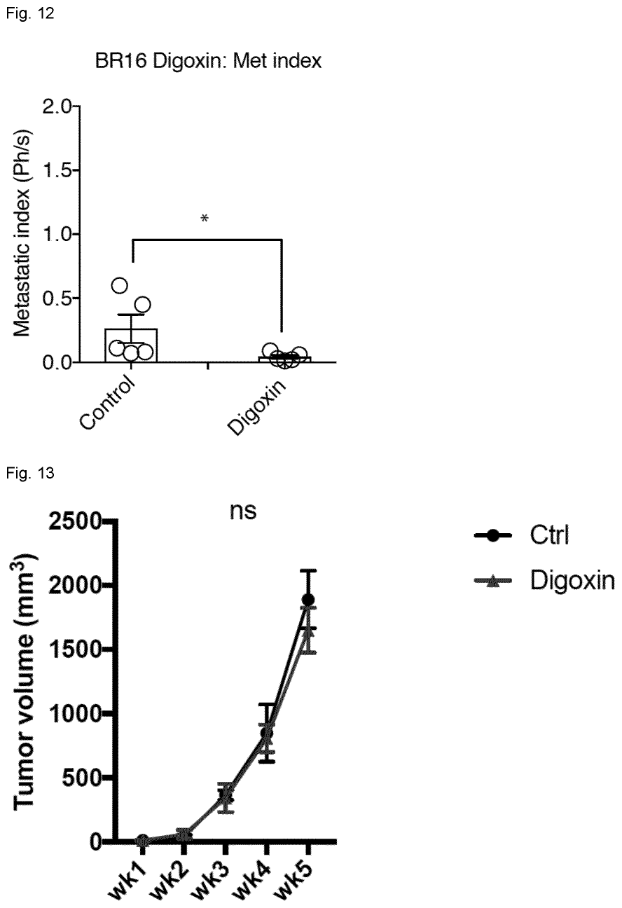

For digoxin treatment in BR16 xenograft mice, no significant difference in tumor size was observed (FIG. 10). However, digoxin treatment results in a clear decrease in the number of CTC clusters and CTC-neutrophil clusters (FIG. 11) and suppresses metastasis (FIG. 12).

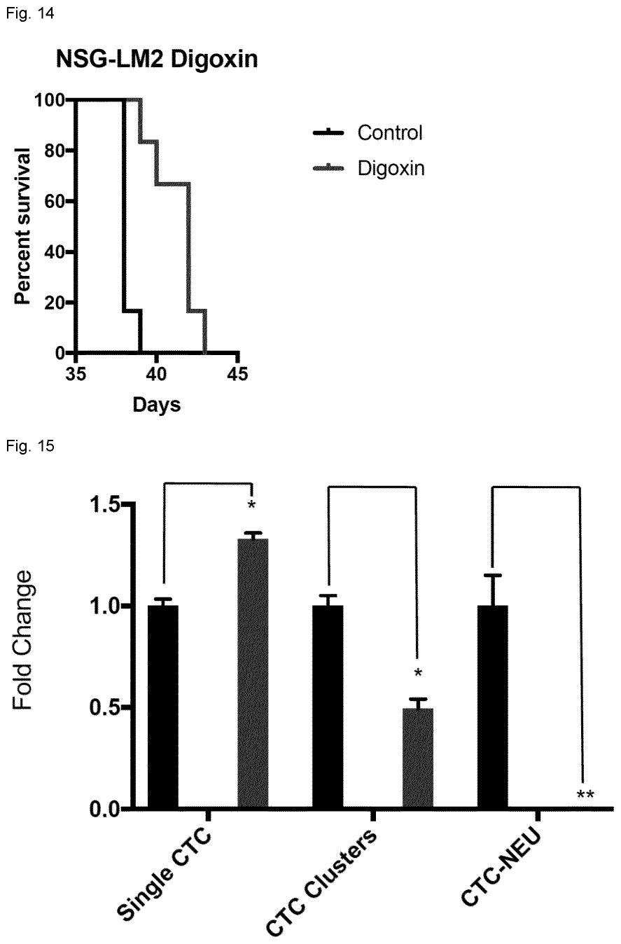

Similarly, for treatment in LM2 xenograft mice, no significant difference in tumor size was observed (FIG. 13). Yet, digoxin treatment prolonged overall survival (FIG. 14) and reduced the formation of CTC clusters and CTC-neutrophil clusters (FIG. 15).

Together, these results demonstrate that Na⁺/K⁺ ATPase inhibition in vivo suppresses the ability of a cancerous lesion to spontaneously shed CTC clusters, leading to a remarkable reduction in metastasis seeding ability.

### Clinical Trial

Patients will receive a daily maintenance dose of digoxin. The daily dose of digoxin will be calculated according to the renal function and the target serum digoxin concentration and applied in an adjusted regimen based on the availability of 0.125 mg and 0.25 mg pills in the morning (before 10 am). Blood samples for analyses of mean CTC cluster size will be drawn at screening, on day 0 (2 hrs after first oral intake), on day 3 and on day 7. Depending on the digoxin serum level maintenance therapy with digoxin will be continued up to 3 weeks if the digoxin serum level on day 7 or day 14 is below 0.70 ng/ml. For the third week of maintenance therapy individual dose adjustments will be carried out as needed.

### Material and Methods

### Cell Culture

CTC derived cells were maintained under hypoxia (5% oxygen) on ultra low attachment (ULA) 6-well plates (Corning, Cat #3471-COR). CTC growth medium containing 20 ng/ml recombinant human Epidermal Growth Factor (Gibco, Cat #PHG0313), 20 ng/ml recombinant human Fibroblast Growth Factor (Gibco, Cat #100-18B), 1×B27 supplement (Invitrogen, Cat #17504-044) and 1× Antibiotic-Antimycotic (Invitrogen, Cat #15240062) in RPMI 1640 Medium (Invitrogen, Cat #52400-025) was added every third day. For passaging, cells were spun down at 800 g for 5 min using a Heraeus Multifuge X3R centrifuge (Invitrogen, Cat #75004515). The supernatant was subsequently aspirated and cells were resuspended in 2 ml/well CTC medium and plated in 6-well ULA plates. BR 16 CTC-derived cells were generated in the inventors lab. Brx50 CTC-derived cells were obtained from the Haber and Maheswaran lab (MGH Cancer Center, Harvard Medical School, Boston, Mass.). MDA-MB-231 (LM2) cells were donated from Joan Massague's lab (MSKCC, New York, N.Y., USA) and passaged in DMEM/F-12 medium (Invitrogen, Cat #11330057) supplemented with 10% FBS (Invitrogen, Cat #10500064) and 1× Antibiotic-Antimycotic (Invitrogen, Cat #15240062). For passaging, LM2 cells were washed once with D-PBS (Invitrogen, Cat #14190169) and dissociated using 0.25% Trypsin (Invitrogen, Cat #25200056).

### CTC Capture and Identification

Blood specimens for CTC analysis were obtained from University Hospital Basel after informed patient consent according to protocol EKNZ BASEC 2016-00067 and EK 321/10, which received ethical approval from the Swiss authorities (EKNZ, Ethics Committee northwest/central Switzerland). An average of 7.5 ml of blood per patient was drawn in EDTA vacutainers. Within 1 hr from blood draw, the blood was processed through Parsortix GEN3D6.5 Cell Separation Cassette (Angle Europe). For mouse studies, blood was retrieved via cardiac puncture and 1 ml of blood was similarly processed through a Parsortix device. Captured CTCs were further stained on Parsortix cassette with EpCAM-AF488 conjugated (CellSignaling, Cat #CST5198), HER2-AF488 (#324410, BioLegend), EGFR-FITC conjugated (GeneTex, Cat #GTX11400) and CD45-BV605 conjugated (Biolegend, Cat #304042 (anti-human); Cat #103140 (anti-mouse)) antibodies. For all other models (xenografts), carrying cancer cells stably expressing a GFP-Luciferase reporter, only anti-CD45 staining was performed, while CTCs were identified based on GFP expression. The number of captured CTCs, including single CTCs, CTC clusters and CTC-WBC clusters, was determined while cells were still in the cassette. CTCs were then released from the cassette in DPBS (#14190169, Gibco) onto ultra-low attachment plates (#3471-COR, Corning). Representative pictures were taken at 40× magnification with Leica DM14000 fluorescent microscope using Leica LAS and analyzed with ImageJ.

### Differential White Blood Cell Staining on CTC-WBC Clusters

Live CTCs captured within the Parsortix microfluidic cassette were stained with anti-Biotin-CD45 (#103104, BioLegend) and detected with Streptavidin-BV421 (#405226, BioLegend), anti-mouse Ly-6G-AF594 (#127636, BioLegend) and anti-CD11b-AF647 (clone M1/70, kind gift from Dr. Roxane Tussiwand, University of Basel) or with anti-F4/80-AF594 (#123140, BioLegend) and CD11b-AF647. Additionally, MMTV-PyMT-derived CTCs were marked with EpCAM-AF488 (#118210, BioLegend). Next, cells were gently released from the microfluidic system into ultra-low attachment plate and immediately imaged (Leica DM1400). The number of CTC-WBC-clusters with neutrophils (Ly-6G+CD11 b^{med}), monocytes (Ly-6G⁻CD11b^{med/high}) and macrophages (F4/80⁺CD11b⁺) was assessed. Immediately after imaging, cells were centrifuged (500 rpm, 3 minutes) on a glass slide and fixed in methanol for 1 minute. After brief air-drying, slides were stained using Wright-Giemsa stain kit (#9990710, ThermoFisher) to visualize nuclear morphology of captured cells, following the manufacturer's instructions.

### Tumorigenesis Assays

All mouse experiments were carried out in compliance with institutional guidelines.

For tail vein experiments, NOD SCID Gamma (NSG) mice (Jackson Labs) were injected with 1×10⁶ BR16-mCherry cells resuspended in 100 μl D-PBS and monitored with IVIS Lumina II (Perkin Elmer). For CTC xenograft mouse model isolation, 1×10⁶ LM2-GFP, 1×10⁶ BRx50-GFP or 1×10⁶ BR16-GFP cells were resuspended in 100 μl of 50% Cultrex PathClear Reduced Growth Factor Basement Membrane Extract (R&D Biosystems, Cat #3533-010-02) in D-PBS and injected orthotopically in NSG mice. Blood draw was performed 4-5 weeks after tumor onset for LM2 cells, 5-6 months after tumor onset for BR16 and 6-7 months after tumor onset for BRx50 cells.

### Single-Cell Micromanipulation

Enriched CTCs were harvested from Parsortix cassette in 1 ml D-PBS solution (Invitrogen, Cat #14190169) in a 6-well ultra low attachment plate (Corning, Cat #3471-COR) and visualized using a CKX41 Olympus inverted fluorescent microscope (part of the AVISO CellCelector Micromanipulator—ALS). Single CTCs and CTC clusters were identified based on intact cellular morphology, AF488/FITC-positive staining and lack of BV605 staining. Target cells were individually micromanipulated with a 30 μM glass capillary on the AVISO CellCelector micromanipulator (ALS) and deposited into individual PCR tubes (Axygen, Cat #321-032-501) containing 10 μl of 2× Digestion Buffer (EZ DNA Methylation Direct Kit-Zymo, Cat #D5020) for WGBS or 2 μl of RLT lysis buffer (Qiagen, Cat #79216) supplemented with 1 U/μl SUPERase In RNAse inhibitor (Invitrogen, Cat #AM2694) for RNA sequencing, and immediately flash frozen in liquid nitrogen.

### Single Cell Whole-Genome Bisulfite Sequencing

Proteinase K digestion and bisulfite treatment was performed according to manufacturer's instructions for EZ DNA Methylation Direct Kit (Zymo, Cat #D5020). Bisulfite-treated DNA was eluted using 9 μl of Elution Buffer and used for library generation with TruSeq DNA methylation kit (Illumina, Cat #EGMK91396) according to manufacturer's instructions. For amplification, 18 cycles were performed using Failsafe Enzyme (Illumina, Cat #FSE51100) and indexes were introduced with Index Primers' Kit (Illumina, Cat #EGIDX81312). Library purification was performed using Agencourt AMPure XP beads at a ratio of 1:1 according to manufacturer's instructions. To avoid DNA loss during pipetting steps, Corning DeckWork low binding barrier pipet tips were used (Sigma, Cat #CLS4135-4X960EA). Library concentration was estimated using Qubit DS DNA HS Assay Kit according to manufacturer's instructions (Invitrogen, Cat #Q32854).

### RNA-Seq Library Generation

RNA was captured on beads conjugated with oligo-dT primer according to Macaulay et al. (Nat Protoc 11, 2081-2103, 2016). cDNA was generated according to Picelli at al.'s Smart-Seq 2 protocol (Nat Protoc 9, 171-181, 2014). Sequencing libraries were generated and indexed from 0.25 ng of cDNA per sample using the Nextera XT DNA Library Preparation Kit (Illumina, Cat #FC-131-2001) according to manufacturer's instructions

### FDA-Approved Compound Screen

A library containing 2,486 FDA-approved compounds was purchased from the Nexus Platform ETH Zurich. Each compound was resuspended using CTC medium at a 15 μM concentration and 20 μl were aliquoted in duplicate in a total of 64 Flat Bottom Clear Ultra Low attachment 96-well plates (Corning, Cat #3474).

To reduce cluster size in CTC derived cell lines, a 40 μm cell strainer was used (Corning, Cat #431750). 40 μl containing 5′000 CTC-derived cells were seeded per well in 96-well ultra low attachment plates that contained 20 μl of pre-aliquoted FDA-approved compounds at 15 μM concentration, so that final compound concentration was 5 μM. Plates were incubated in hypoxia (5% oxygen) for 2 days and then 20 μl were transferred into a 96 well Black/clear Tissue culture treated plate (BD Falcon, Cat #353219) containing 40 μl of D-PBS (Invitrogen, Cat #14190169) and stained for 1 hr at 37° C. with a final concentration of 4 μM Hoechst 34580 (Invitrogen, Cat #H21486), 2 μM TMRM (Invitrogen, Cat #T668) and 4 μM TOTO-3 (Invitrogen, Cat #T3604). For each plate, two positive controls (non-treated cells) and two negative controls (non-treated and 40 μM-filtered cells) were included. Z-factors were calculated per individual plate using the following formula: Z′=1−3(σₛ+σ_{c})/|μₛ−μ_{c}|³ (σ: standard deviation, μ: mean, s: positive control and c: negative control) (Martin et al., PLoS One 9, e88338, 2014) and ranged between 0.62-0.937. Plates were scanned using Operetta High Content Imaging System (Perkin Elmer) and cluster analysis was performed using Harmony High Content Imaging and Analysis Software (Perkin Elmer).

### Enrichment Scores

An enrichment score (ES) indicates the over- or underrepresentation of a certain object within a sample of many objects (=enrichment). A positive ES indicates that a certain feature is overrepresented as compared to other features within an analysed set of features (=enrichment). A negative enrichment score indicates the opposite, namely that a feature is less present than to be expected by the values of other features in the sample. In other words, a positive ES for a transcription factor binding site (TFBS) indicates that the TFBS is represented in the sample to a higher degree than other TFBS (=enriched). An enrichment score can be normalized by dividing a specific ES by the mean of the enrichment scores for all objects in the dataset to yield a normalized enrichment score (NES). Normalization of the enrichment score accounts for differences in gene set size and in correlations between gene sets and the expression dataset; therefore, a normalized enrichment scores (NES) can be used to compare analysis results across gene sets. Only TFBS with a NES score are considered significant shown in the analysis.

### CRISPR-CAS9 CLDN3/4 Double Knock Out in BR16

The inventors used lentiviral delivery of pLenti-Cas9-EGFP vector (Addgene) to generate a BR16 CTC-derived cell line that stably expresses the Cas9 protein together with GFP. In BR16-Cas9-GFP line the inventors then introduced sgRNA sequences that target either CLDN3 or CLDN4. In detail, sgRNA sequences were designed using the GPP Web Portal (https://portals.broadinstitute.org/gpp/public/analysis-tools/sgrna-design). Two sgRNAs targeting CLDN3 ((sense) 5′-CACGTCGCAGAACATCTGGG-3′ (SEQ ID NO 01) and (sense) 5′-ACGTCGCAGAACATCTGGGA-3′; (SEQ ID NO 02)) were cloned in vector pLentiGuide-Puro (Addgene) and 2 sgRNAs targeting CLDN4 ((sense) 5′-CAAGGCCAAGACCATGATCG-3′ (SEQ ID NO 03) and (sense) 5′-ATGGGTGCCTCGCTCTACGT-3′; (SEQ ID NO 04)) were cloned in vector pLentiGuide-Blast. Vector pLentiGuide-Blast was generated by replacing puromycin resistance gene on plasmid pLentiGuide-Puro with the blasticidin resistance gene using the MluI and BsiWI restriction enzyme sites. Double positive-clones were selected based on puromycin (1 μg/mL) and blasticidin (10 μg/mL) antibiotic selection for 2 weeks and CLDN3/CLDN4 knockout was verified by western blot.

### Survival Analyses

Survival analyses were performed using the survival R package (v 2.41-3). Kaplan-Meier curves were generated and Log-Rank test was used to estimate the significance of the difference in survival between groups. For patients, progression-free survival was defined as the period between primary tumor diagnosis and first relapse. For mouse model analysis, death was selected as the endpoint for the analysis and defined as the moment a given animal had to be euthanized according to the inventors' mouse protocol guidelines.

1. A method for treatment of metastasis in cancer, comprising administering to a patient in need thereof an Na⁺/K⁺ ATPase inhibitor, thereby treating the metastasis.

2. The method according to claim 1, wherein the inhibitor is a cardiac glycoside.

3. The method according to claim 2, wherein the cardiac glycoside is selected from a cardenolide and a bufadienolide.

4. The method according to claim 2, wherein the cardiac glycoside is selected from digitoxin, ouabain, convallatoxin, proscillaridin, lanatoside C, gitoformate, peruvoside, strophanthidin, metildigoxin, deslanoside, bufalin, digoxin and digoxigenin.

5. The method according to claim 2, wherein the cardiac glycoside is selected from digoxin, digitoxin and ouabain, particularly wherein the cardiac glycoside is digoxin.

6. The method according to claim 5, wherein the cardiac glycoside is digoxin and wherein a daily dose of digoxin is 0.125 mg to 0.25 mg.

7. The method according to claim 5, wherein the cardiac glycoside is digoxin and wherein a digoxin serum level is adjusted to between 0.70 ng/ml and 1.0 ng/ml.

8. The method according to claim 1, wherein the Na⁺/K⁺ ATPase inhibitor is effective in the disruption of CTC clusters.

9. A method for treatment of venous thromboembolism associated with cancer comprising, administering to a patient in need thereof an Na+/K+ ATPase inhibitor, thereby treating the venous thromboembolism.

10. The method according to claim 1, wherein the cancer is breast cancer or prostate cancer.

11. A method for treatment of metastatic cancer or for treatment of venous thromboembolism in cancer patients comprising, administering to a patient in need thereof a nucleic acid molecule comprising an inhibitor nucleic acid sequence capable of downregulating or inhibiting expression of a target nucleic acid sequence encoding a protein selected from:
CLDN3,
CLDN4 and
Na+/K+ ATPase or any of its constituent subunit isoforms,
thereby treating the metastatic cancer or venous thromboembolism.

12. The method of claim 11, wherein said inhibitor nucleic acid sequence is able to specifically hybridize with a sequence or subsequence of
an exon comprised in said target nucleic acid sequence,
an intron comprised in said target nucleic acid sequence,
a promoter region modulating expression of said target nucleic acid sequence, and/or
an auxiliary sequence regulating expression of said target nucleic acid sequence.

13. The method of claim 11, wherein said inhibitor nucleic acid sequence is an antisense oligonucleotide, an siRNA, an shRNA, an sgRNA or an miRNA.

14. The method according to claim 11, wherein the inhibitor nucleic acid sequence comprises or consists of nucleoside analogues.

15. The method according to claim 11, wherein the cancer is breast cancer or prostate cancer.

16. The method according to claim 9, wherein the cancer is breast cancer or prostate cancer.


SEQUENCE LISTING (4 sequences)

SEQ ID NO: 1 - DNA, 20 bp, Artificial Sequence
  sgRNA
cacgtcgcagaacatctggg

SEQ ID NO: 2 - DNA, 20 bp, Artificial Sequence
  sgRNA
acgtcgcagaacatctggga

SEQ ID NO: 3 - DNA, 20 bp, Artificial Sequence
  sgRNA
caaggccaagaccatgatcg

SEQ ID NO: 4 - DNA, 20 bp, Artificial Sequence
  sgRNA
atgggtgcctcgctctacgt